(19) United States
(12) Patent Application Publication
(10) Pub. No.: US 2025/0244212 A1
(43) Pub. Date: Jul. 31, 2025

(54) METHODS AND SYSTEMS FOR IMAGING BIOPSY SAMPLES

(71) Applicant: University of Washington, Seattle, WA (US)

(72) Inventors: Eric J. Seibel, Seattle, WA (US); Matthew Damien Carson, Seattle, WA (US)

(73) Assignee: University of Washington, Seattle, WA (US)

(21) Appl. No.: 18/717,921

(22) PCT Filed: Dec. 7, 2022
(86) PCT No.: PCT/US2022/052192

§ 371 Date: Jun. 7, 2024

Related U.S. Application Data
(60) Provisional application No. 63/287,464, filed on Dec. 8, 2021.

(51) Int. Cl. G01N 1/31; G01N 1/30; G01N 21/64; G01N 33/50; G01N 35/00

(52) U.S. Cl. CPC G01N 1/31; G01N 21/64; G01N 33/5005; G01N 35/00871; G01N 2001/305; G01N 2800/7028

(57) ABSTRACT

This disclosure relates to methods and systems for imaging biopsy samples. The system for imaging biopsy samples comprises a window and an imaging device. The window comprises an inflexible and optically clear material. The window is configured to compress a sample disposed in a biopsy needle. The imaging device is configured to maintain a constant working distance to the flattened tissue surface while scanning. The imaging device is configured to capture one or more images of the sample through the window.

### CROSS-REFERENCE TO RELATED APPLICATION(S)

This application is a U.S. National Phase Application which claims the priority of International Application No. PCT/US2022/052192, titled METHODS AND SYSTEMS FOR IMAGING BIOPSY SAMPLES″, and filed Dec. 7, 2022. Application No. PCT/US2022/052192 claims the priority of U.S. Provisional App. No. 63/287,464, titled “SYSTEM AND METHOD FOR DIAGNOSTIC IMAGING OF A BIOPSY FROM A CORING NEEDLE” and filed on Dec. 8, 2021, and which is incorporated by reference herein in its entirety.

### STATEMENT REGARDING FEDERALLY SPONSORED RESEARCH OR DEVELOPMENT

This invention was made with government support under Grant No. R21CA246359-02 awarded by the National Institutes of Health. The government has certain rights in the invention.

### TECHNICAL FIELD

This application relates to medical devices, and in particular, to methods and systems for imaging biopsy samples.

### BACKGROUND

In the field of pathological diagnosis, tissue samples collected from recipients are useful for making diagnostic decisions and determining a stage of disease progression. A biopsy is a type of procedure used to collect tissue samples. Once removed from a recipient, a tissue sample can be stained and reviewed by a trained pathologist. For instance, the pathologist can determine whether the recipient has a particular type of cancer by staining and reviewing the tissue sample.

In many cases, the pathologist reviews the tissue sample hours or days after the tissue sample is obtained. During this delay, the cells in the tissue sample may die, atrophy, or otherwise degrade. To preserve the physiology of the tissue sample during this delay, the tissue sample is typically placed in a chemical preservative within a short time after being removed from the recipient. After preservation, the tissue sample may be transported to the pathologist. In many cases, the tissue sampled is stored for an extended period of time before the pathologist reviews the sample. Transporting and storing the tissue sample is time-consuming and costly. Moreover, there is a risk that the tissue sample can be lost, broken, or degraded during transportation or storage.

In some cases, a rapid on-site evaluation (ROSE) of a tissue sample can be conducted by a trained professional shortly after a biopsy is performed. Using ROSE, the trained professional can provide preliminary diagnostic information. However, clinicians in many low-resource settings are unable to perform ROSE.

### SUMMARY

Various systems and methods disclosed and contemplated herein relate to methods and systems for imaging biopsy samples.

Throughout this disclosure, the term “biopsy sample,” and its equivalents, may refer to a tissue sample obtained from a subject, such as a sample obtained using a biopsy procedure. A biopsy device can be any types of devices that are suitable for collecting and transferring biopsy samples from a patient or a recipient, such as a sheathed needle (e.g., an end-cut needle, a side-cut needle, or the like), a fine needle, a biopsy punch, or the like. A recipient can be an animal, a human, a plant, a living object, or the like. An imaging device can include, but is not limited to, an optical microscope, an electron microscope, a stereomicroscope, a florescence microscope, or any combination thereof. Tissue stains or dyes may include, but are not limited to, hematoxylin and eosin (H&E), Hoechst, rhodamine, or the like. The inflexible and optically clear material includes, but is not limited to, glass, plastic, quartz, crystal, sapphire, or the like. The elastic material includes, but is not limited to, silicone, nylon, latex, rubber, polyester, sealed fabric, or the like. An adhesive that makes a chemical bonding to glass or quartz includes, but is not limited to, silane (e.g., XIAMETER OFS-6040 silane produced by EM Corporation), epoxies, or the like.

Throughout this disclosure, a microfluidic circuit that includes reservoirs and pumps are discussed. Microfluidics is a multidisciplinary field that involves engineering, physics, chemistry, biochemistry, nanotechnology, and biotechnology. Principles and theories of microfluidics have practical applications in various technologies, such as inkjet printheads, DNA chips, lab-on-a-chip technology, micro-propulsion, and micro-thermal technologies. Researchers or users can implement microfluidics theory to control and manipulation of fluids that are geometrically constrained to a small scale (typically sub-millimeter).

Techniques discussed here provide a biopsy imaging system which includes an imaging device and a container. The container can receive a biopsy device that may contain one or more biopsy samples. The container can include a window that is suitable for exposing the biopsy sample(s) to the imaging device. The imaging device can be configured to capture images of the biopsy sample(s) in the container via the window.

In some examples, the window can include a flexible component and a transparent component. The imaging device can contact the transparent component via a support assembly and apply a pressing force on the surface of the transparent component of the window. The support assembly can be made of metal or any suitable material. The pressing force can be implemented mechanically (e.g., by a motor, actuator, etc.) or manually (e.g., by a manually operated handle or press, etc.). As such, the flexible component can deform in response to the force applied by the imaging device. The transparent component can be pressed to compress the biopsy sample(s). The imaging device can scan and take images of the biopsy sample(s) while applying the force on the surface of the transparent component of the window.

Additionally or alternatively, the window can be made of rigid and optically clear material without the flexible component. In such a case, the imaging device can apply a pressing force to a rigid window and the window can contact and compress the biopsy sample. In some examples, the imaging device can move along various directions (e.g., horizontally, vertically, laterally, or the like) while applying the pressing force to the window. The imaging device can capture images of different parts of the biopsy sample(s) while scanning the window.

In some examples, the biopsy imaging system can further include a staining mechanism (not shown) configured to provide a staining solution to stain the biopsy sample(s) in the container. The staining solution may contain one or more tissue stains or dyes that enhance contrast for morphological imaging and molecular imaging.

In some examples, the biopsy imaging system can further include a transportation mechanism on which the container and/or the biopsy device can be placed. The transportation mechanism can move in various directions to adjust the position of the imaging device, the container, the biopsy device, or a combination thereof, such that the biopsy sample(s) can be stained and imaged. In some examples, the transportation mechanism can include one or more actuators (e.g., electric motors, stepper motors, jackscrews, or the like) configured to move the imaging device and/or the container along various directions.

In some examples, the biopsy imaging system can implement a machine learned model to provide assistant diagnostic information based, at least in part, on the images of the biopsy sample(s). In some examples, the assistant diagnostic information can include information regarding whether the cells of the biopsy sample(s) include pathologies such as at least one cancer cell, the degree of invasiveness of the disease, cancer phenotype, or the like. As used herein, the terms “machine learned model,” “machine learning model,” and their equivalents, may refer to a computer-based algorithm configured to identify patterns in training data, and to recognize those patterns in additional data. In some instances, the machine learned model can include any suitable models, algorithms, and/or machine learning algorithms. For example, the machine learned model in the memory can be implemented as a neural network. As described herein, an example neural network is a biologically inspired algorithm that passes input data through a series of connected layers to produce an output. Each layer in a neural network can also include another neural network, or can include any number of layers (whether convolutional or not). As can be understood in the context of this disclosure, a neural network can utilize machine learning, which can refer to a broad class of such algorithms in which an output is generated based on learned parameters.

The imaging device generates image data by detecting light reflected and/or emitted from the biopsy sample(s) in the biopsy device. The biopsy imaging system can be configured to store, aggregate, process, and/or transmit the image data generated by the imaging device. The biopsy imaging system can be in communication with a remote computing system for example, via the one or more networks. The biopsy imaging system can transmit the image data, the assistant diagnostic information, and other data to the remote computing system. The remote computing system can be configured to receive, display, and/or analyze the data from the biopsy imaging system. For example, the remote computing system can include a display configured to visually output images of the biopsy sample(s) based on the data. The remote computing system can also display the assistant diagnostic information based on the data. The remote computing system can be associated with a user. In some examples, the display can display a user interface (UI) which may facilitate an interaction between the remote computing system and the user. The user can view the images of the biopsy sample(s), the assistant diagnostic information, and other data via the UI. The user can also provide feedback, diagnostic comments, opinions, or the like via the Ul through the remote computing system. In some examples, the user can be a pathologist, an oncologist, a cytologist, a physician, or another type of care provider. For example, the user can determine that the sample includes pathologies, such as cancer cells.

### BRIEF DESCRIPTION OF THE DRAWINGS

The following figures, which form a part of this disclosure, are illustrative of the described technology and are not meant to limit the scope of the claims in any manner.

FIG. 1 shows a schematic block diagram of an example environment for imaging and analyzing biopsy samples in accordance with implementations of this disclosure.

FIG. 2A and FIG. 2B illustrates an example system for imaging biopsy samples in accordance with implementations of this disclosure.

FIG. 3A illustrates a side view of a system for imaging biopsy samples in accordance with implementations of this disclosure, where a biopsy device carrying biopsy sample(s) is placed into a container of the system.

FIG. 3B illustrates a side view of the system for imaging biopsy samples in accordance with implementations of this disclosure, where a sheath of the biopsy device is retrieved and the biopsy sample(s) is exposed.

FIG. 3C illustrates a side view of the system for imaging biopsy samples in accordance with implementations of this disclosure, where a sheath of the biopsy device is retrieved, and the biopsy sample(s) is exposed.

FIG. 3D illustrates a side view of the system for imaging biopsy samples in accordance with implementations of this disclosure, where the imaging device applies a pressing force on the window of the container to compress the biopsy sample(s).

FIG. 3E illustrates an end view of the system for imaging biopsy samples in accordance with implementations of this disclosure, where the imaging device applies a pressing force on the window of the container to compress the biopsy sample(s).

FIG. 3F illustrates a top view of the system for imaging biopsy samples in accordance with implementations of this disclosure, where the biopsy samples are flushed out of the container by wash fluid.

FIG. 4A illustrates a side view of a system for imaging biopsy samples in accordance with implementations of this disclosure, where a biopsy device carrying biopsy sample(s) is placed into a container of the system.

FIG. 4B illustrates a side view of the system for imaging biopsy samples in accordance with implementations of this disclosure, where a sheath of the biopsy device is retrieved, and the biopsy sample(s) is exposed.

FIG. 4C illustrates a side view of the system for imaging biopsy samples in accordance with implementations of this disclosure, where a sheath of the biopsy device is retrieved, and the biopsy sample(s) is exposed.

FIG. 4D illustrates a side view of the system for imaging biopsy samples in accordance with implementations of this disclosure, where the compressing component applies a pressing force on the first window of the container to compress the biopsy sample(s).

FIG. 4E illustrates a side view of the system or imaging biopsy samples in accordance with implementations of this disclosure, where the compressing component applies a pressing force on the first window of the container, and the imaging device applies a force on the second window of the container to compress the biopsy sample(s).

FIG. 4F illustrates a top view of the system for imaging biopsy samples in accordance with implementations of this disclosure, where the biopsy sample(s) is flushed out.

FIG. 5 illustrates a side view of an example system for imaging biopsy samples in accordance with implementations of this disclosure, where a container of the system for imaging biopsy samples comprises two windows.

FIG. 6 illustrates a side view of an example system for imaging biopsy samples in accordance with implementations of this disclosure, where a container of the system for imaging biopsy samples comprises two windows, and the needle of the biopsy device is retrieved.

FIG. 7A and FIG. 7B illustrate side views of another example biopsy imaging system in accordance with implementations of this disclosure.

FIG. 8 illustrates a side view of another example system for imaging biopsy samples in accordance with implementations of this disclosure.

FIG. 9A and FIG. 9B illustrate an example system for imaging biopsy samples in accordance with implementations of this disclosure.

FIG. 10 illustrates a flow chart of an example process for imaging biopsy samples in accordance with implementations of this disclosure.

FIG. 11 illustrates an example system configured to enable and/or perform the some or all of the functionality discussed herein.

### DETAILED DESCRIPTION

The techniques described herein may be implemented in a number of ways. Example implementations are provided below with reference to the following figures. Although discussed in the context of pathological imaging, the methods, apparatuses, and systems described herein may be applied to a variety of imaging systems, and are not limited to the imaging of pathological samples.

Existing techniques for preparing and evaluating tissue samples, such as those obtained from biopsies, have several shortcomings. First, current techniques that include transporting and storing tissue samples are time-consuming. These techniques produce unnecessary delays in diagnosis, resulting in delays in patient care. These delays can also be very stressful for the patients themselves, who may have to wait for days before knowing whether they have a serious illness. Second, current techniques requiring the assistance of trained pathologists with particular equipment are relatively expensive. Accordingly, these techniques are not feasible for low-resource settings, such as health clinics located in rural settings or developing nations.

Various implementations of the present disclosure address these and other problems by providing techniques for efficiently evaluating tissue samples shortly after they are obtained from a recipient. In some instances, the preparation procedure can be conducted at the point of care or at the bedside where the biopsy is performed. High-quality images of the tissue sample can be acquired and reviewed immediately after the tissue sample is obtained. In some cases, the images are transmitted to a remote reviewer (e.g., a pathologist, a cytopathologist, a cytologist, or other specialists) who can make a diagnostic and/or therapeutic decision.

A core needle biopsy (CNB) procedure is an example of a minimally-invasive method of removing a tissue sample from the recipient. Such a procedure is performed in order to determine if the tissue from which the sample is obtained is indicative of a pathology, such as cancer. For example, a CNB can be performed on a mass in the breast, liver, or pancreas of the recipient, and a pathologist reviewing the sample can deduce whether the mass is cancerous. A rapid on-site evaluation procedure can be performed using a tissue sample obtained via a CNB. For instance, a care provider can manually remove the small thread of tissue from the needle (e.g., roughly 1 to 2 millimeters in diameter), touching, rolling, and/or squashing the tissue against a microscope slide to remove cells from the outer surface. The cells of the tissue adhering to the slide can be rapidly fixed, stained, and imaged under a microscope. A trained professional (such as a cytopathologist or cytologist) can provide a preliminary diagnosis of cancer at the bedside by viewing the sample under the microscope. Such a procedure can also be referred to as “Touch Prep.” Although this procedure can produce rapid preliminary results, it has been reported to be damaging to the integrity of the sample for further downstream analysis by a pathologist.

Currently, there is no clinical procedure to perform a rapid on-site evaluation using CNBs that is not damaging to the biopsy. Furthermore, there is no clinical rapid on-site evaluation procedure that images the biopsy sample (such as tissue or the like) so that more than a diagnosis of cancer cells can be made, such as the degree of invasiveness of cancer which can be determined from images of tissue, not isolated cells.

The clinical importance of making a preliminary diagnosis using a tissue biopsy is growing with the advancement of therapies that have specific molecular targets. Determining the degree of invasiveness and cancer phenotype during the biopsy procedure can provide more rapid feedback to the patient, physician, and oncologist. Such feedback can affect the biopsy procedure, such as performing additional CNBs for molecular tests. In the case of conventional histopathology, a complete disease diagnosis may take several days to determine a treatment strategy, delaying the start of treatment by weeks. In environments with limited medical professionals and infrastructure, such delays can be months, and occasionally the patient never receives lifesaving information and/or treatments.

There is an existing unmet need for a rapid on-site tissue evaluation at the point of care when the biopsy is procured. A non-destructive method of determining the presence of disease and more rapidly establishing a strategy for treatments can be implemented if the procedure does not affect subsequent conventional histopathology. A time window of approximately 3 to 5 minutes is available for the rapid on-site tissue evaluation conducted on intact tissue samples at the bedside. In some examples, the procedure can be automated and the results can be communicated to a remote pathologist or trained image-analysis system. The operation of the rapid on-site tissue evaluation procedure can be conducted by a wider range of individuals who are not necessarily trained in tissue analysis, such as interventional radiologists, nurses, or healthcare technicians at the patient's bedside.

Various embodiments of the present disclosure will be described in detail with reference to the drawings, wherein like reference numerals represent like parts and assemblies throughout the several views. Additionally, any examples set forth in this specification are not intended to be limiting and merely set forth some of the many possible embodiments.

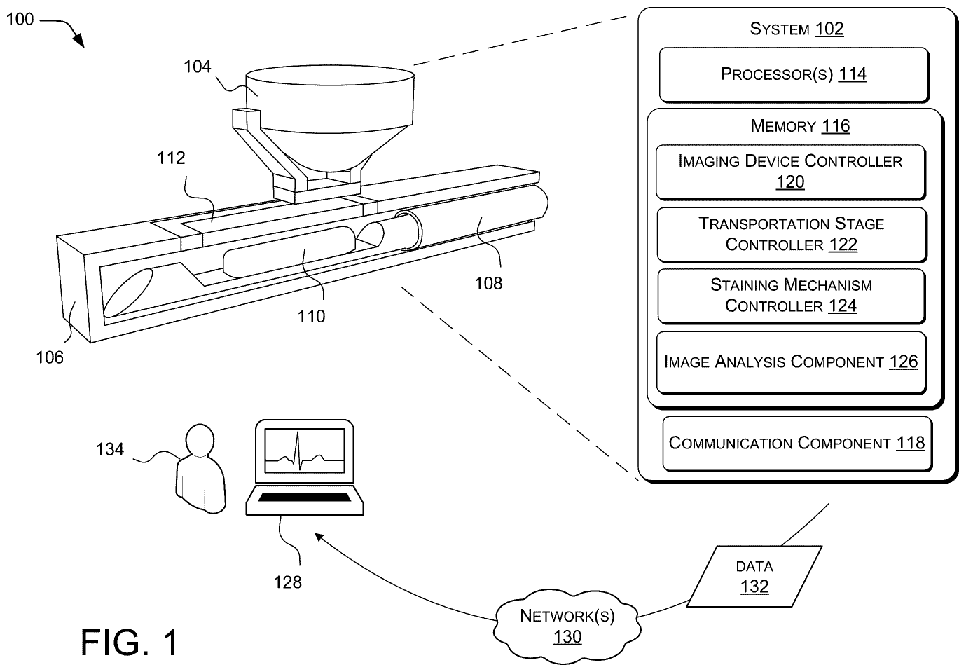

FIG. 1 shows a schematic block diagram of an example environment 100 for imaging and analyzing biopsy samples in accordance with implementations of this disclosure. As shown in FIG. 1, an example biopsy imaging system 102 includes an imaging device 104 and a container 106. The container 106 can receive a biopsy device 108 which may contain one or more biopsy samples 110. The container 106 can include a window 112 which is suitable for exposing the biopsy sample(s) 110 to the imaging device 104. The imaging device 104 can be configured to capture images of the biopsy sample(s) 110 in the container 106 via the window 112.

The biopsy imaging system 102 can include a microfluidic circuit which includes a first reservoir (not shown), a second reservoir (not shown), the container 106, at least one waste receptacle (not shown), pumps, tubing (not shown) connecting the first reservoir, the second reservoir, the container 106, at least one waste receptacle, and pumps. The first reservoir is configured to store a tissue stain configured to stain cells. The second reservoir is configured to store a wash fluid. The at least one waste receptacle can be configured to collect the tissue stain and/or the wash fluid. The pumps are configured to move the tissue stain from the first reservoir into the container 106, and move the tissue stain out of the container 106 and into the at least one waste receptacle. The pumps are further configured to move a wash fluid from the second reservoir into the container 106, and move the wash fluid out of the container 106 and into the at least one waste receptacle.

The biopsy imaging system 102 can further include a staining mechanism (not shown) configured to provide a staining solution to stain the biopsy sample(s) 110 in the container 106. The staining solution may contain one or more tissue stains or dyes that enhance contrast for morphological imaging and molecular imaging. In some examples, the staining solution may include different types of stains or dyes that are useful for diagnosing different types of pathologies, such as different types of cancers.

The biopsy imaging system 102 can further include a transportation mechanism (not shown) on which the container 106 and/or the biopsy device 108 can be placed. In some examples, the transportation mechanism can move in various directions to adjust the position of the imaging device 104, the container 106, the biopsy device 108, or a combination thereof, such that the biopsy sample(s) 110 can be stained and imaged. In some examples, the transportation mechanism can include one or more actuators (e.g., electric motors, stepper motors, jackscrews, or the like) configured to move the imaging device 104 and/or the container 106 along various directions.

In various implementations, the imaging device 104 generates image data by detecting light reflected and/or emitted from the biopsy sample(s) 110 in the biopsy device 108. The biopsy imaging system 102 can be configured to store, aggregate, process, and/or transmit the image data generated by the imaging device 104. The system 102 includes one or more processors 114, memory 116, and a communication component 118. The processor(s) 114 can be a single processing unit or a number of units, each of which could include multiple different processing units. The processor(s) 114 can include a microprocessor, a microcomputer, a microcontroller, a digital signal processor, a central processing unit (CPU), a graphics processing unit (GPU), a security processor, etc. Alternatively, or in addition, some or all of the techniques described herein can be performed, at least in part, by one or more hardware logic components. For example, and without limitation, illustrative types of hardware logic components that can be used include a Field-Programmable Gate Array (FPGA), an Application-Specific Integrated Circuit (ASIC), an Application-Specific Standard Products (ASSP), a state machine, a Complex Programmable Logic Device (CPLD), pulse counters, resistor/coil readers, other logic circuitry, a system on chip (SoC), and/or any other devices that perform operations based on instructions. Among other capabilities, the processor(s) 114 can be configured to fetch and execute computer-readable instructions stored in the memory 116.

The memory 116 can include one or a combination of computer-readable media. As used herein, “computer-readable media” includes computer storage media and communication media. Computer storage media includes volatile and non-volatile, removable, and non-removable media implemented in any method or technology for the storage of information, such as computer-readable instructions, data structures, program modules, or other data. Computer storage media includes, but is not limited to, Phase Change Memory (PRAM), Static Random-Access Memory (SRAM), Dynamic Random-Access Memory (DRAM), other types of Random-Access Memory (RAM), Read Only Memory (ROM), Electrically Erasable Programmable ROM (EEPROM), flash memory or other memory technology, Compact Disk ROM (CD-ROM), Digital Versatile Discs (DVD) or other optical storage, magnetic cassettes, magnetic tape, magnetic disk storage or other magnetic storage devices, or any other medium that can be used to store information for access by a computing device. In contrast, communication media includes computer-readable instructions, data structures, program modules, or other data in a modulated data signal, such as a carrier wave. As defined herein, computer storage media does not include communication media. In some examples, non-transitory computer-readable media does not include communication media. In some examples, the memory 116 can include an operating system configured to manage hardware and services within and coupled to a device for the benefit of other modules, components, and devices.

The system 102 can include a communication component 118 configured to communicate with other computing device(s) and/or to communicate via the network(s) 130. In some examples, the communication component 118 can transmit data using one or more protocols or languages, such as an extensible markup language (XML), Modbus, Hypertext Transfer Protocol (HTTP), Hypertext Transfer Protocol Secure (HTTPS), Universal Serial Bus (USB), etc. In some examples, the communication component 118 includes at least one transceiver configured to transmit data wirelessly to an external device.

The memory 116 can include one or more computer-executable modules (components) that are executable by the processor(s) 114 to perform functions. In some examples, the memory 116 can include an imaging device controller 120 configured to control the imaging device 104 to capture images. For example, the system 102 can include one or more actuators (e.g., electric motors, stepper motors, jackscrews, or the like) configured to move the imaging device 104 and/or the container 106 along various directions. The imaging device controller 120 can also be configured to move the imaging device 104 in various directions to adjust the position of the imaging device 104. The imaging device controller 120 can also be configured to control the imaging device 104 to apply pressing force to the window 112. The imaging device controller 120 can also be configured to control the force applied to the window 112 with sensors (such as vision sensors, displacement sensors, and/or force sensors).

The memory 116 can include a transportation mechanism controller 122 configured to control the transportation mechanism to move along various directions (e.g., horizontally, vertically, diagonally, or the like). In some examples, the container 106 and/or the biopsy device 108 can be placed on the transportation mechanism to move together with the transportation mechanism. The transportation mechanism controller can be configured to control the transportation mechanism to change the position of the container 106 and/or the biopsy device 108 for staining and/or imaging the biopsy sample(s) 110.

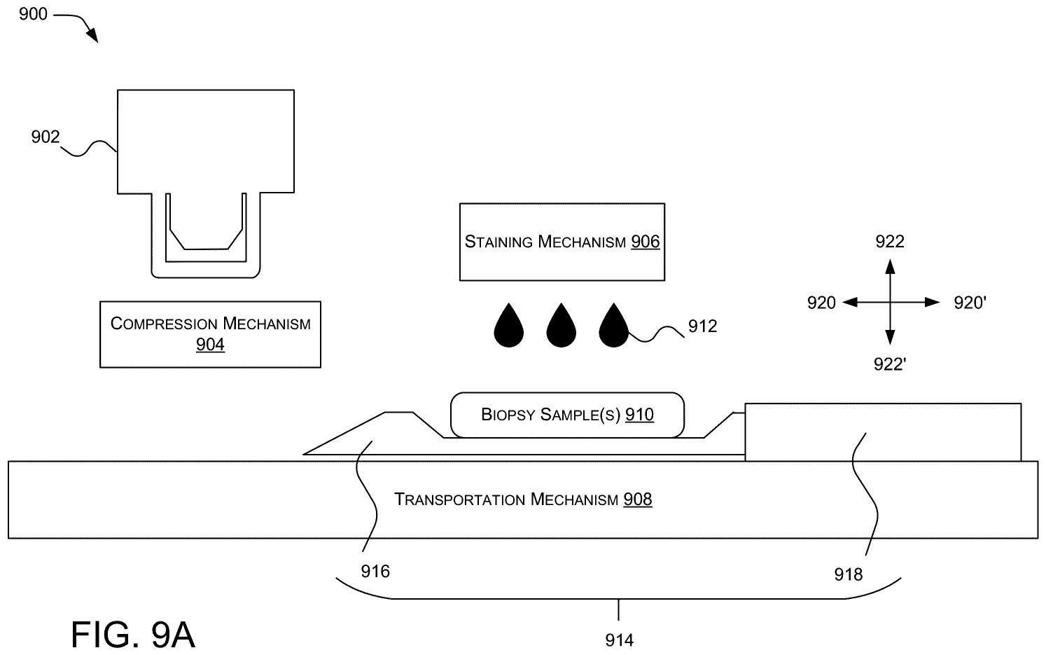

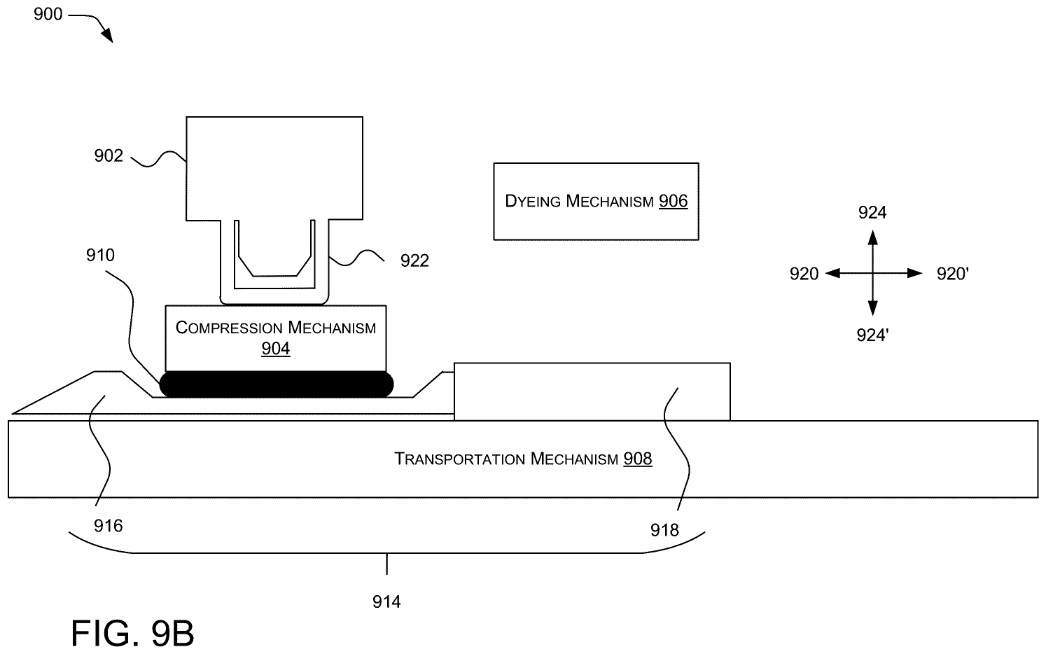

The memory 116 can include a staining mechanism controller 124 configured to control the staining mechanism to stain the biopsy sample(s) 110. For example, the staining mechanism controller 124 can be configured to control the staining mechanism to provide the staining solution to the container 106 via one or more ports (not shown) at a given fluidic rate. The staining mechanism controller 124 can also be configured to control the staining mechanism to discharge the staining solution from the container 106 after the biopsy sample(s) 110 is stained. Additionally or alternatively, the staining mechanism controller 124 can be configured to dispense the staining solution (such as in the form of droplets or a spray) to the biopsy sample(s) 110 rather than immersing the biopsy sample(s) 110. Additional details are described with respect to FIG. 9A. For example, the staining mechanism controller 124 can be configured to dribble the staining solution on the surface of the biopsy sample(s) 110. The biopsy sample(s) 110 can be stained by dribbling drops of the staining solution along the needle of the biopsy device which carries the biopsy sample(s) 110. For example, the staining solution can be dribbled in a single drop to the biopsy sample(s) 110, followed by another drop to the biopsy sample(s) 110 to provide a little overlap. Next, a wash fluid can be provided to the container 106 to rinse the biopsy sample(s) 110. The wash fluid can also rinse the container 106 and the contents thereof. As such, the staining solution can be washed away before the images are captured. In some examples, the wash fluid can include one or more of saline solution, formalin, or alcohol chemical fixation, or other ingredients. As such, the image quality of the biopsy sample(s) can be improved when the biopsy sample(s) is later pressed against the window. In some examples, the wash fluid can be dribbled on the biopsy sample(s) 110. For example, the wash fluid can be sprayed (more force than dribbling) into the biopsy sample(s) 110 to rinse the excess staining solution from the biopsy sample(s) 110 before imaging. Alternatively or additionally, the wash fluid can be introduced into the container 106 to flush the container 106. In some instances, the container 106 can be rinsed with the wash fluid multiple times.

The memory 116 can include an image analysis component 126 configured to provide assistant diagnostic information based, at least in part, on the images of the biopsy sample(s) 110. In some examples, the assistant diagnostic information can include information regarding whether the cells of the biopsy sample(s) 110 include pathologies such as at least one cancer cell, the degree of invasiveness of the disease, cancer phenotype, or the like. In some examples, the image analysis component 126 can implement a machine learned model to analyze the images of the biopsy sample(s) 110. As used herein, the terms “machine learned model,” “machine learning model,” and their equivalents, may refer to a computer-based algorithm configured to identify patterns in training data, and to recognize those patterns in additional data. In some instances, the machine learned model can include any suitable models, algorithms, and/or machine learning algorithms. For example, the machine learned model in the memory 116 can be implemented as a neural network. As described herein, an example neural network is a biologically inspired algorithm that passes input data through a series of connected layers to produce an output. Each layer in a neural network can also include another neural network, or can include any number of layers (whether convolutional or not). As can be understood in the context of this disclosure, a neural network can utilize machine learning, which can refer to a broad class of such algorithms in which an output is generated based on learned parameters.

Although discussed in the context of neural networks, any type of machine learning can be used consistently with this disclosure. For example, machine learning algorithms can include, but are not limited to, regression algorithms (e.g., ordinary least squares regression (OLSR), linear regression, logistic regression, stepwise regression, multivariate adaptive regression splines (MARS), locally estimated scatterplot smoothing (LOESS)), instance-based algorithms (e.g., ridge regression, least absolute shrinkage and selection operator (LASSO), elastic net, least-angle regression (LARS)), decisions tree algorithms (e.g., classification and regression tree (CART), iterative dichotomiser 3 (ID3), Chi-squared automatic interaction detection (CHAID), decision stump, conditional decision trees), Bayesian algorithms (e.g., naïve Bayes, Gaussian naïve Bayes, multinomial naïve Bayes, average one-dependence estimators (AODE), Bayesian belief network (BNN), Bayesian networks), clustering algorithms (e.g., k-means, k-medians, expectation maximization (EM), hierarchical clustering), association rule learning algorithms (e.g., perceptron, back-propagation, hopfield network, Radial Basis Function Network (RBFN)), deep learning algorithms (e.g., Deep Boltzmann Machine (DBM), Deep Belief Networks (DBN), Convolutional Neural Network (CNN), Stacked Auto-Encoders), Dimensionality Reduction Algorithms (e.g., Principal Component Analysis (PCA), Principal Component Regression (PCR), Partial Least Squares Regression (PLSR), Sammon Mapping, Multidimensional Scaling (MDS), Projection Pursuit, Linear Discriminant Analysis (LDA), Mixture Discriminant Analysis (MDA), Quadratic Discriminant Analysis (QDA), Flexible Discriminant Analysis (FDA)), Ensemble Algorithms (e.g., Boosting, Bootstrapped Aggregation (Bagging), AdaBoost, Stacked Generalization (blending), Gradient Boosting Machines (GBM), Gradient Boosted Regression Trees (GBRT), Random Forest), SVM (support vector machine), supervised learning, unsupervised learning, semi-supervised learning, etc.

The biopsy imaging system 102 can be in communication with a remote computing system 128, for example, via the one or more networks 130. For example, the biopsy imaging system 102 can transmit data 132 to the remote computing system 128. The data 132 can include the images of the biopsy samples(s) 110 captured by the imaging device 104, the assistant diagnostic information generated by the image analysis component, and other data. In some examples, other data can include, but is not limited to, time data (e.g., the time at which the imaging device 104 captured the images), position data (e.g., a position of the imaging device 104 and/or the container 106 at the time the imaging device 104 captured the images), position data of the transportation mechanism (e.g., x, y, z coordinates of the transportation mechanism, or the like), stain type (e.g., which one or more stains were used to stain the biopsy sample(s) 110), or the like.

The networks 130 can be any type of wireless network or other communication network known in the art. Examples of the networks 130 include the Internet, an intranet, a wide area network (WAN), a local area network (LAN), and a virtual private network (VPN), cellular network connections, and connections made using protocols such as Institute of Electrical and Electronics Engineers (IEEE) standards, including 802.11a, b, g, n, and/or ac. The 802.11a, b, g, n, and/or ac are IEEE standards used for wireless routers, Wi-Fi access points, and Wi-Fi in portable devices.

The remote computing system 128 can be configured to receive, display, and/or analyze the data 132. For example, the remote computing system 128 can include a display configured to visually output images of the biopsy sample(s) 110 based on the data 132. The remote computing system 128 can also display the assistant diagnostic information based on the data 132. The remote computing system 128 can be associated with a user 134. In some examples, the display can display a user interface (UI) which may facilitate an interaction between the remote computing system 128 and the user 134. The user 134 can view the images of the biopsy sample(s) 110, the assistant diagnostic information, and other data. The user 134 can also provide feedback, diagnostic comments, opinions, or the like via the Ul through the remote computing system 128. In some examples, the user 134 can be a pathologist, an oncologist, a cytologist, a physician, or another type of care provider. For example, the user 134 can determine that the sample includes pathologies, such as cancer cells.

The techniques described herein may be performed by various devices in a medical environment, such as bedside devices, care on point devices, test systems, and so forth.

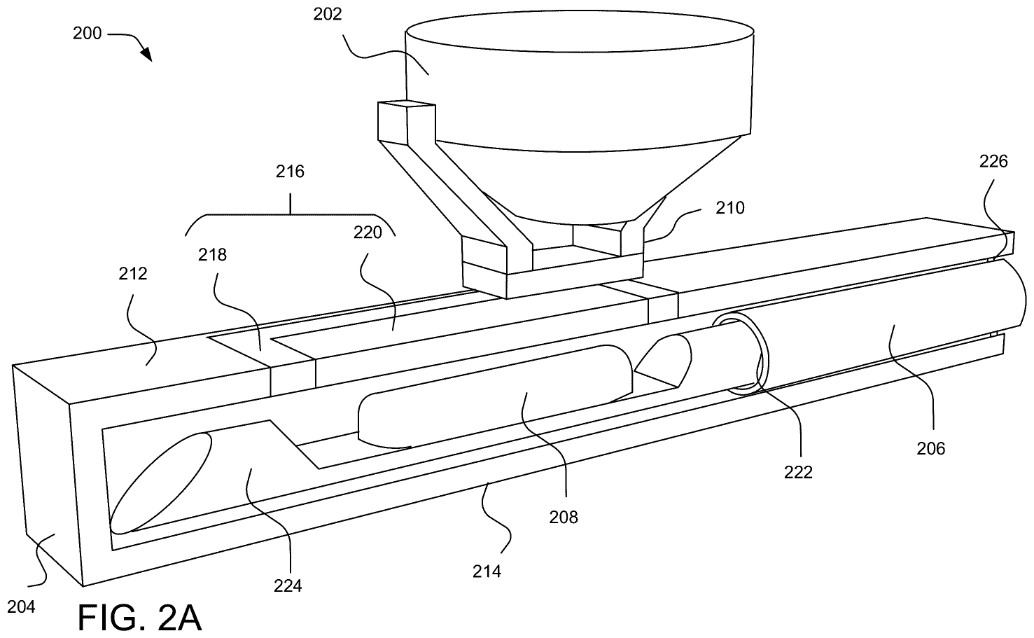

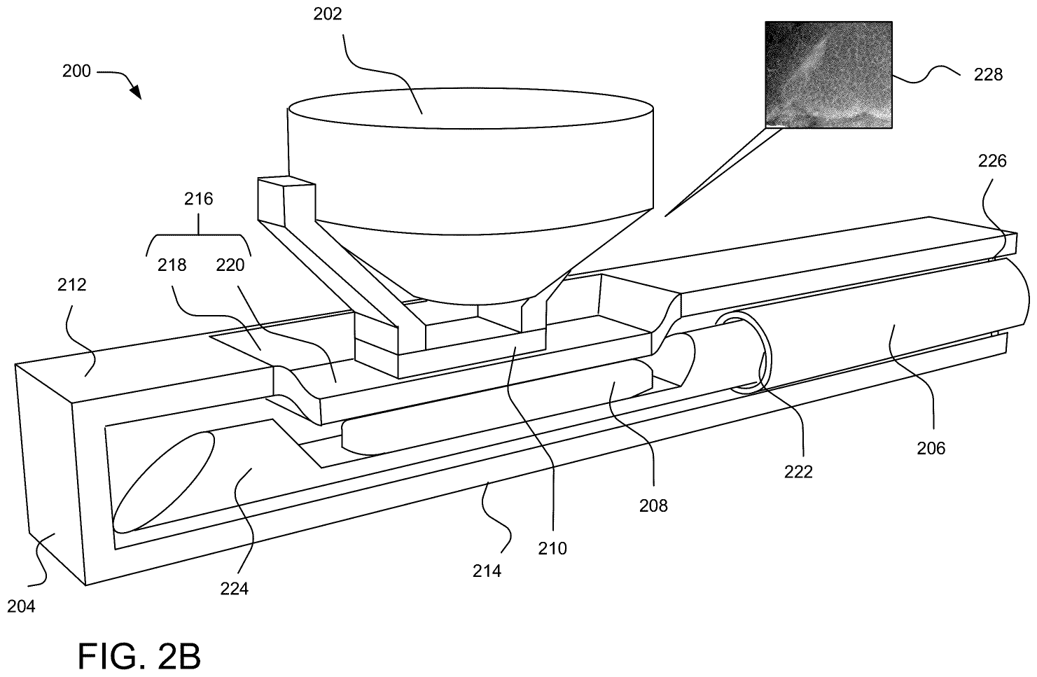

FIG. 2A and FIG. 2B illustrates an example system 200 for imaging biopsy samples in accordance with implementations of this disclosure. As shown in FIG. 2A, the system 200 for imaging biopsy samples includes an imaging device 202 and a container 204, where the container 204 can receive a biopsy device 206 which may contain the biopsy sample(s) 208.

The imaging device 202 can include, but is not limited to, a light microscope, an electron microscope, a fluorescence microscope, a digital microscope, a stereoscopic microscope, or the like. The imaging device 202 can further include a camera (not shown) such as an optical camera, a digital camera, an infrared camera, or the like. The imaging device 202 can further include a support assembly 210 configured to compress the container 204 and/or a soft tissue sample disposed inside the container 204.

The container 204 includes an imaging side 212 arranged proximate to the imaging device 202. The imaging side 212 faces the imaging device 202. The container 204 further includes an opposite side 214 which is opposite to the imaging side 212. In some examples, the container 204 includes a window 216 on the imaging side. Though FIG. 2A shows that the window 216 has a rectangular shape, the window 216 can have other shapes, such as a round shape, a polygon shape, an oval shape, an irregular shape, or the like. The window 216 includes a flexible component 218 and a transparent component 220. An outer edge of the flexible component 218 can be coupled to an inner edge of the window 216. For example, the outer edge of the flexible component 218 can be adhered to the inner edge of the window 216 using an adhesive that makes a chemical bonding to glass or quartz such as silane (e.g., XIAMETER OFS-6040 silane produced by EM Corporation), epoxies, or the like. An outer edge of the transparent component 220 can be coupled to an inner edge of the flexible component 218.

The flexible component 218 can be made of elastic material. In some examples, the flexible component 218 can have a ring shape, a bangle shape, a frame shape, a folded bellow shape, or any other shape that can accommodate the transparent component 220. In some examples, the flexible component 218 can be coupled to the window 216 and the main portion of the container 204 in a fluid-tight (e.g., watertight) manner, such that a fluid in the container 204 is held inside the container 204. The flexible component 218 can be configured to accommodate the transparent component 220.

The transparent component 220 can be made of an inflexible and optically clear material. In some examples, the transparent component 220 can be made of materials that allow light to travel therethrough. Additionally or alternatively, the transparent component 220 can be made of materials that allow electron beam, infrared, ultraviolet, electromagnetic wave, fluorescence, or the like to travel therethrough. Though FIG. 2A shows that the transparent component 220 has a rectangular shape, the transparent component 220 can have other shapes, such as a round shape, a polygon shape, an oval shape, an irregular shape, or the like. The size of the transparent component 220 can be configured to expose the entire biopsy sample. For example, the length of the transparent component 220 can be 20 mm, 25 mm, 30 mm, or the like. For example, the width of the transparent component 220 can be 1 mm, 2 mm, 3 mm, 5 mm, or the like.

In this example, the biopsy device 206 can be a sheathed needle which includes a sheath 222 and a needle 224. The sheath 222 can be configured to accommodate the needle 224. The needle 224 can be configured to collect and preserve biopsy samples 208 from a patient or a recipient. In some examples, a recipient can be an animal, a human, a plant, a living object, or the like.

The biopsy device 206 can contain one or more biopsy samples 208. The biopsy device 206 can be inserted into the container 204. The sheath 222 can be retrieved to expose the needle 224 such that the biopsy samples 208 can be exposed to the imaging side 212 of the container 204. The needle 224 can be positioned such that the biopsy samples 208 are under the transparent component 220 of the window 216 and suitable for being imaged by the imaging device 202 through the window 216. In some examples, the biopsy sample(s) 208 can include, but is not limited to, soft tissues, clumps of cells, or the like.

The biopsy sample(s) 208 can be stained with a staining solution. The staining solution can be introduced into the container 204 via one or more ports (not shown) at given fluidic rates. The staining solution may contain one or more tissue stains or dyes that enhance contrast for morphological imaging and/or molecular imaging. In some examples, the staining solution may be suitable to stain nuclei. In some examples, the tissue stains or dyes may include, but are not limited to, hematoxylin and eosin (H&E), Hoechst stain, rhodamine, acid fuchsine, iodine, methylene blue, or the like. In some examples, the staining solution can include various types of stains or dyes. For example, various types of stains or dyes in the staining solution can be useful to diagnose different types of pathologies, such as different types of cancers.

In some implementations, the staining solution includes an immunostain. For instance, the staining solution includes an antibody that specifically binds to a predetermined antigen. In some cases, the presence and/or amount of the antigen in the biopsy sample(s) 208 is indicative of whether the biopsy sample(s) 208 expresses a particular pathology. For example, the antigen could be a specific protein expressed by a predetermined type of cancer cell. In some implementations, the antibody is conjugated to an enzyme that catalyzes a reaction that can be detected by imaging, such as a color-changing reaction. In some cases, the antibody is tagged to a fluorophore (e.g., fluorescein) that fluoresces in response to receiving excitation light. According to various implementations, an indirect immunohistochemistry technique is utilized, such that a first antibody specifically binds to the antigen and a second antibody specifically binds to the first antibody, wherein the second antibody is attached to the enzyme or fluorophore. Alternatives to antibodies are using peptides that bind with high affinity and specificity, such as human epidermal growth factor receptor 2 (HER-2) and epidermal growth factor receptor (EGFR), which can be conjugated to fluorescence or colorimetric dyes or nanoparticles.

After the biopsy sample(s) 208 is stained, the staining solution can be discharged from the container 204 via the one or more ports. In some examples, the one or more ports can include exit ports and entrance ports, where the exit ports are configured to discharge the staining solution out of the container 204, and entrance ports are configured to introduce the staining solution into the container 204. Additionally or alternatively, the one or more ports can be used for both introducing and discharging the staining solution. In some examples, a respective port can have a valve configured to open, close, and control the flow rate of the respective port.

The container 204 can further include a sealing component 226 configured to seal the container 204 such that the container 204 is fluid-tight. The sealing component 226 is disposed between the biopsy device 206 and the container 204, and is configured to seal fluid (such as a staining solution, a wash fluid, or the like) inside the container 204. The sealing component 226 can be configured to accommodate the biopsy device 206 when the biopsy device 206 is introduced into the container 304. In some examples, the sealing component 226 can be made of silicone, rubber, polytetrafluoroethylene (PTFE), fluorosilicone (FVMQ), polyurethane, or the like.

Referring to FIG. 2B, the imaging device 202 can compress the window 216 via the support assembly 210. The imaging device 202 can contact the transparent component 220 via the support assembly 210 and apply force on the surface of the transparent component 220. As such, the flexible component 218 can deform in response to the force applied by the imaging device 202. The transparent component 220 can be pressed towards the biopsy sample(s) 208 against the needle 224. Though in this example, the biopsy sample(s) 208 is placed on the needle 224, in some examples, the sample 208 can be placed directly in the container 204. The biopsy sample(s) 208 can be compressed by the transparent component 220 due to the force applied by the imaging device 202. Then, the imaging device 202 can capture images of the biopsy sample(s) 208. In some examples, the imaging device 202 can move along various directions (e.g., horizontally, vertically, laterally, or the like) while applying the pressing force to the surface of the transparent component 220 within or outside the field of view of the imaging device 202. The imaging device 202 can capture images of different parts of the biopsy sample(s) 208 while moving along various directions.

In some examples, the imaging device 202 can further include a storage component (not shown) configured to store the images of the biopsy sample(s) 208. The storage component includes volatile and non-volatile, removable, and non-removable media implemented in any method or technology for the storage of information, such as computer-readable instructions, data structures, program modules, or other data. The storage component includes, but is not limited to, Phase Change Memory (PRAM), Static Random-Access Memory (SRAM), Dynamic Random-Access Memory (DRAM), other types of Random-Access Memory (RAM), Read Only Memory (ROM), Electrically Erasable Programmable ROM (EEPROM), flash memory or other memory technology, Compact Disk ROM (CD-ROM), Digital Versatile Discs (DVD) or other optical storage, magnetic cassettes, magnetic tape, magnetic disk storage or other magnetic storage devices, or any other medium that can be used to store information for access by a computing device. In some examples, the memory can include an operating system configured to manage hardware and services within and coupled to a device for the benefit of other modules, components, and devices. Thumbnail image 228 shows an example image of the biopsy sample(s) 208 captured by the imaging device 202 using the method called Microscopy with Ultraviolet Surface Excitation (MUSE).

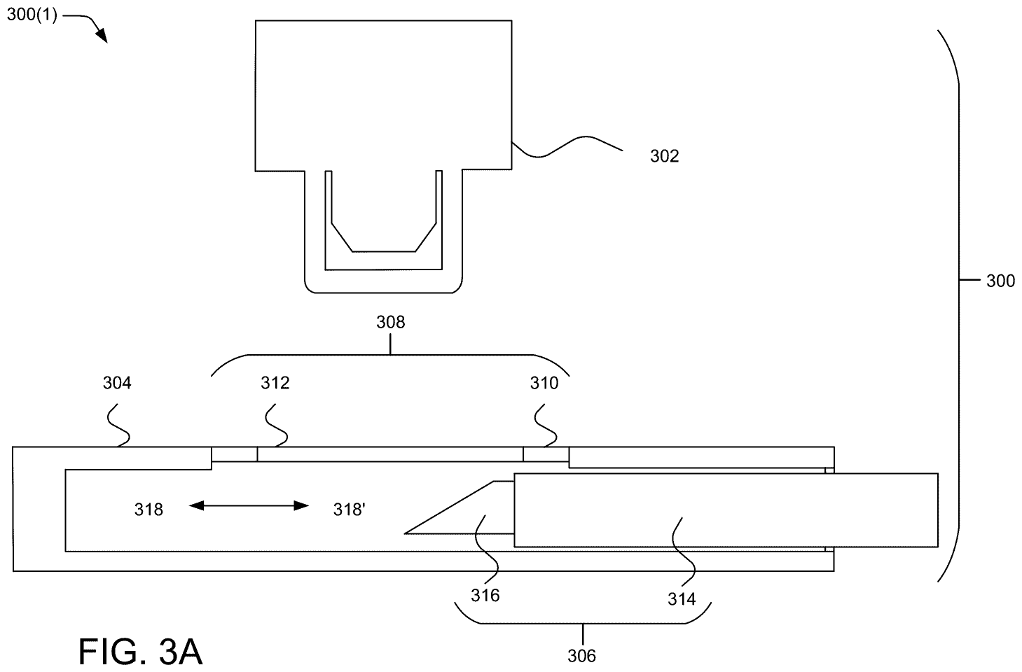

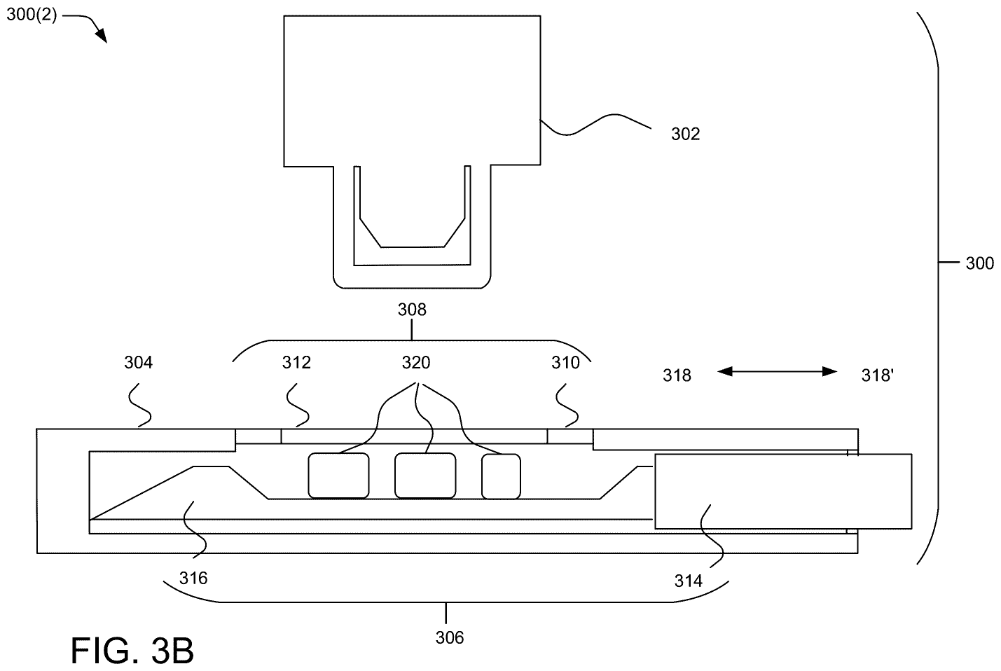

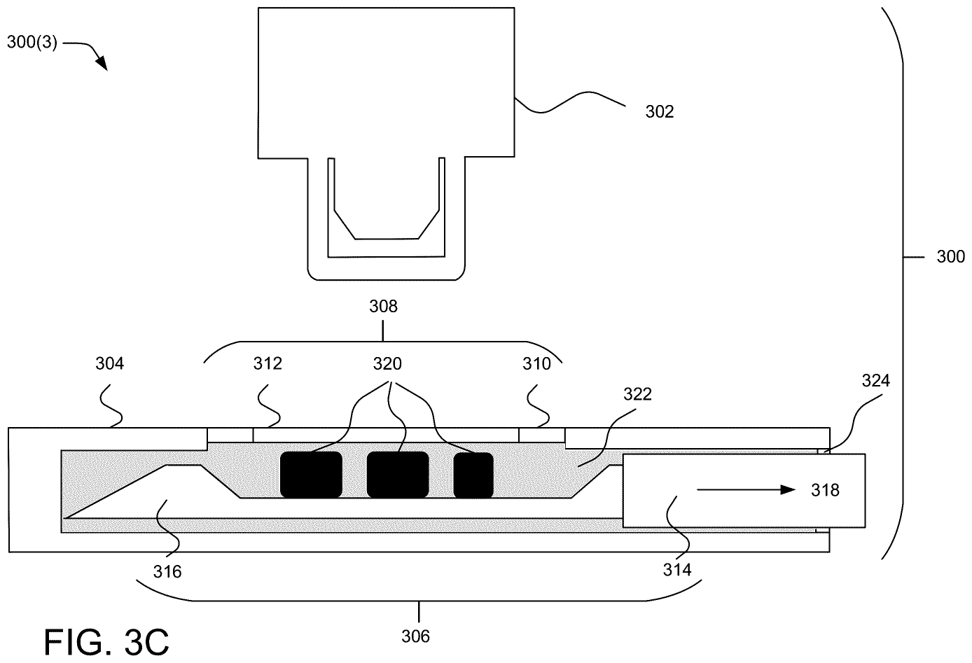

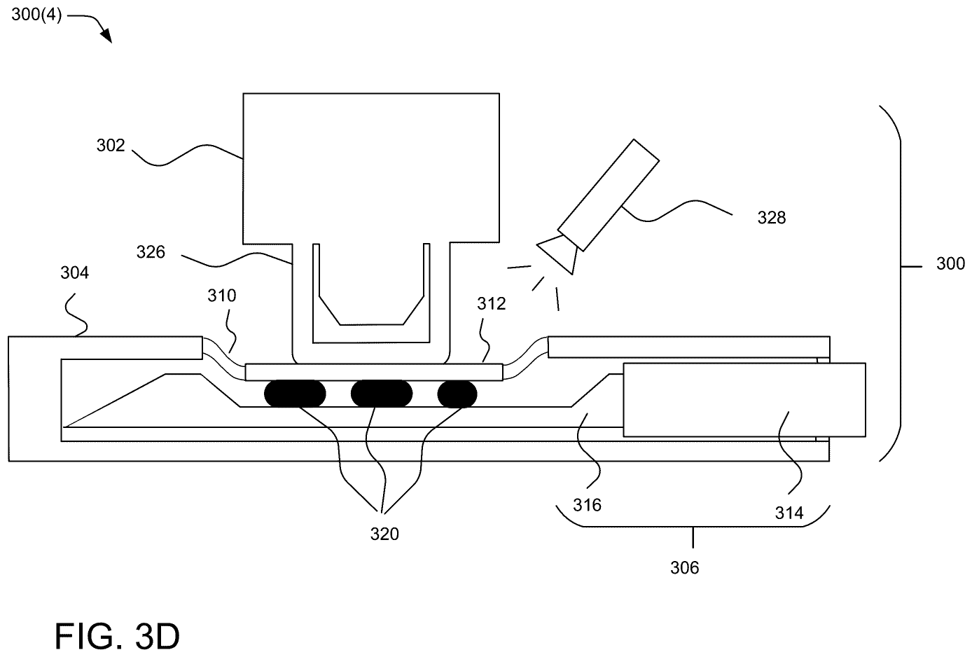

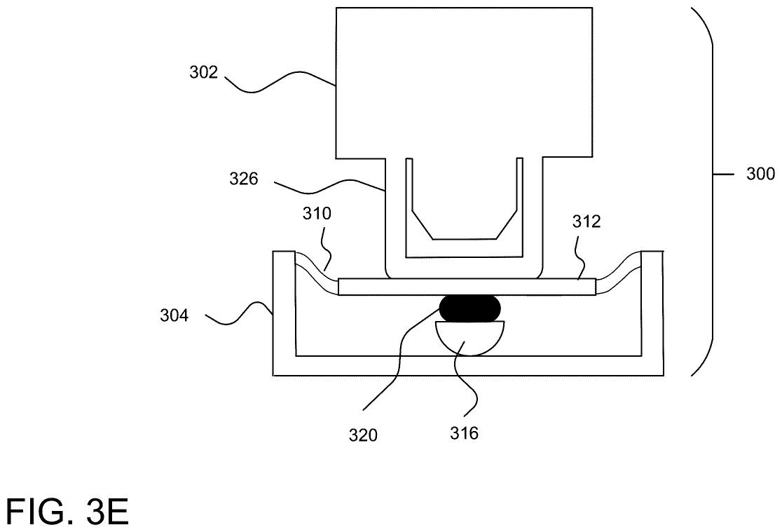

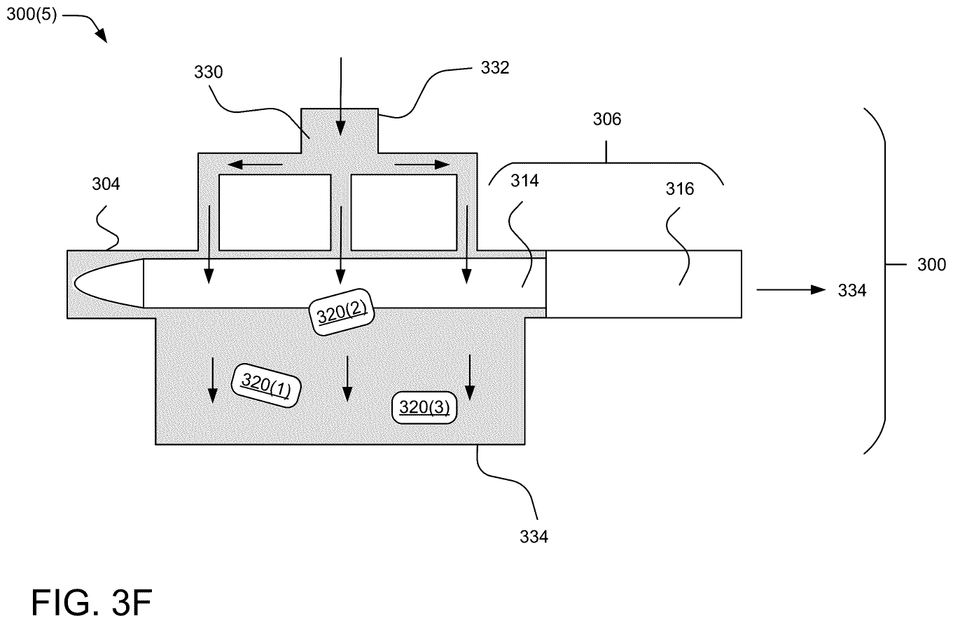

FIG. 3A, FIG. 3B, FIG. 3C, FIG. 3D, FIG. 3E, and FIG. 3F illustrate example operations for imaging biopsy samples using a system 300 for imaging biopsy samples in accordance with implementations of this disclosure. FIG. 3A illustrates a side view of a system 300 for imaging biopsy samples in accordance with implementations of this disclosure, where a biopsy device carrying biopsy sample(s) is placed into a container of the system 300. Referring to FIG. 3A, at operation 300(1), the system 300 for imaging biopsy samples includes an imaging device 302 and a container 304. In some examples, the imaging device 104 can include, but is not limited to, an optical microscope, an electron microscope, a stereomicroscope, a fluorescence microscope, or the like.

The container 304 can receive a biopsy device 306 which may contain one or more biopsy samples. The container 304 can include a window 308 which can include a flexible component 310 and a transparent component 312. An outer edge of the flexible component 310 can be coupled to an inner edge of the window 308. For example, the outer edge of the flexible component 310 can be adhered to the inner edge of the window 308 using an adhesive that makes a chemical bonding to glass or quartz such as silane (e.g., XIAMETER OFS-6040 silane produced by EM Corporation), epoxies, or the like. An outer edge of the transparent component 312 can be coupled to an inner edge of the flexible component 310.

The flexible component 310 can be made of elastic material. In some examples, the flexible component 310 can have a ring shape, a bangle shape, a frame shape, a folded bellow shape, or any other shapes that can accommodate the transparent component 312. In some examples, the flexible component 310 can be watertight. The flexible component 310 can be configured to accommodate the transparent component 312 inside.

The transparent component 312 can be made of an inflexible and optically clear material. In some examples, the transparent component 312 can be made of materials that allow light to travel therethrough. Additionally or alternatively, the transparent component 312 can be made of materials that allow electron beam, infrared, ultraviolet, electromagnetic wave, fluorescence, or the like to travel therethrough. The transparent component 312 can have various shapes, such as a rectangular shape, a round shape, a polygon shape, an oval shape, an irregular shape, or the like. The size of the transparent component 312 can be configured to expose the entire biopsy sample.

In this example, the biopsy device 306 can be a sheathed needle which includes a sheath 314 and a needle 316. The sheath 314 can be configured to accommodate the needle 316. The needle 316 can be configured to collect and preserve biopsy sample(s) 320 from a patient or a recipient. In some examples, a recipient can be an animal, a human, a plant, a living object, or the like. In this example, the biopsy device 306 can be introduced into the container 304 along the direction 318.

FIG. 3B illustrates a side view of the system 300 for imaging biopsy samples in accordance with implementations of this disclosure, where a sheath of the biopsy device is retrieved and the biopsy sample(s) is exposed. Referring to FIG. 3B, at operation 300(2), the sheath 314 of the biopsy device 306 can be retrieved along the direction 318′. The needle 316 of the biopsy device 306 can contain one or more biopsy samples 320. The needle 316 can be placed to expose the biopsy sample(s) 320 to the window 308 which is suitable for exposing the biopsy sample(s) 320 to the imaging device 302. The imaging device 302 can be configured to capture images of the biopsy sample(s) 320 in the container 304 via the window 308.

FIG. 3C illustrates a side view of the system 300 for imaging biopsy samples in accordance with implementations of this disclosure, where a sheath of the biopsy device is retrieved, and the biopsy sample(s) is exposed. Referring to FIG. 3C, at operation 300(3), a staining solution 322 can be introduced into the container 304 to stain the biopsy sample(s) 320. The staining solution 322 may contain one or more tissue stains or dyes that enhance contrast for morphological imaging and molecular imaging. After the biopsy sample(s) 320 is stained, the staining solution can be discharged from the container 304 via the one or more ports. In some examples, the staining solution 322 can stay in the container 204 for a period suitable to stain the biopsy sample(s), for example, 2 minutes, 3 minutes, 5 minutes, or the like. In some examples, the staining solution 322 can include different types of stains.

In some examples, the system 300 for imaging biopsy samples can further include a staining mechanism (not shown) configured to provide the staining solution 322 to the container 304 via one or more ports (not shown). In some examples, the staining solution 322 can be introduced into the container at a given fluidic rate. In some examples, the one or more ports can include exit ports and entrance ports, where the exit ports are configured to discharge the staining solution 322 out of the container 304, and the entrance ports are configured to introduce the staining solution 322 into the container 304. Additionally or alternatively, the one or more ports can be used for both introducing and discharging the staining solution into/from the container 304. In some examples, a respective port can have a valve configured to open, close, and/or control the flow rate of the staining solution 322.

The container 304 can further include a sealing component 324 configured to seal the container 304 such that the container 304 is fluid-tight. The sealing component 324 is disposed between the biopsy device 306 and the container 304, and is configured to seal fluid (such as a staining solution, a wash fluid, or the like) inside the container 304. The sealing component 324 can be configured to accommodate the biopsy device 306 when the biopsy device 306 is introduced into or retrieved out of the container 304. The sealing component 324 that accepts the biopsy device 306 can be a septum or adjustable component that can be clamped to provide a fluid-tight seal while still allowing the sheath 314 to move along the direction 318 and the direction 318′. In some examples, the sealing component 226 can be made of silicone, rubber, polytetrafluoroethylene (PTFE), fluorosilicone (FVMQ), polyurethane, or the like.

In some examples, a fixation reagent can be introduced into the container 304 to fix the biopsy sample(s) 320. Examples of fixation reagents include but are not limited to ethanol, formalin, acetone, picric acid, wax, or the like.

FIG. 3D illustrates a side view of the system 300 for imaging biopsy samples in accordance with implementations of this disclosure, where the imaging device applies a pressing force on the window of the container to compress the biopsy sample(s). Referring to FIG. 3D, at operation 300(4), after the staining solution 322 is discharged from the container 304, the system 300 for imaging biopsy samples can control the imaging device 302 to press, via the support assembly 326, against the transparent component 312 of the window so as to compress the biopsy sample(s) 320. In this example, in response to the pressing force applied by the imaging device 302 via the support assembly 326, the flexible component can deform, and the transparent component 312 can be displaced to press the biopsy sample(s) 320 to compress the biopsy sample(s) 320.

The system 300 for imaging biopsy samples can control the imaging device 302 to capture images of the biopsy sample(s) 320 in the container 304 via the transparent component 312. In some examples, the imaging device 202 can have a focus plane on the surface of the transparent component 312. The focus plane can be adjusted as needed. In some examples, the imaging device 302 can include a wide field-of-view (FOV) objective lens. In some examples, the wide FOV objective lens can be used for a low-magnification wide FOV imaging. Additionally or alternatively, a high-resolution objective lens can be used, while the FOV thereof can be much smaller than the wide FOV objective lens. In some examples, the imaging device 202 can move along various directions (e.g., horizontally, vertically, laterally, or the like) while applying the pressing force to the surface of the transparent component 220. The imaging device 202 can capture images of different parts of the biopsy sample(s) 208 while moving along various directions.

After taking the images of the biopsy sample(s) 320, the imaging device 302 can be lifted from the transparent component 312 of the window. As the force applied to the transparent component 312 goes away, the transparent component 312 can go back to the original position, and the flexible component 310 can recover to the original shape and position.

In some examples, the system 300 for imaging biopsy samples can also include a light source 328 configured to emit light through the transparent component 312. Examples of the light source 328 can include, but are not limited to, a flashlight, lantern, limelight, laser device, light-emitting diode (LED), epi-illumination device, excitation light device, or the like. In some examples in which a biopsy sample is stained with a fluorescent dye, the light source 328 may provide excitation light that causes the fluorescent dye to fluoresce. In some implementations, the system 300 detects the light after it has been reflected from a biopsy sample. In one example, the light source 328 can emit the excitation light which is ultraviolet light. The transparent component 312 can be transparent to ultraviolet light and can be made from quartz, fused silica, or sapphire glass.

FIG. 3E illustrates an end view of the system 300 for imaging biopsy samples in accordance with implementations of this disclosure, where the imaging device applies a pressing force on the window of the container to compress the biopsy sample(s). Referring to FIG. 3D, after the staining solution 322 is discharged from the container 304, the system 300 for imaging biopsy samples can control the imaging device 302 to apply a pressing force, via the support assembly 326, against the transparent component 312 of the window so as to compress the biopsy sample(s) 320. In this example, in response to the pressing force applied by the imaging device 302 via the support assembly 326, the flexible component can deform, and the transparent component 312 can be displaced to press the biopsy sample(s) 320 to compress the biopsy sample(s) 320.

FIG. 3F illustrates a top view of the system 300 for imaging biopsy samples in accordance with implementations of this disclosure, where the biopsy samples are flushed out of the container by wash fluid. Referring to FIG. 3F, at operation 300(5) the system 300 for imaging biopsy samples can introduce wash fluid 330 into the container 304 to flush the container 304 to discharge the biopsy samples 320(1), 320(2), and 320(3). In some examples, the wash fluid can be introduced into the container 304 via an inlet 332. The wash fluid 330 can be introduced into the container 304 at a given fluidic rate. Then the wash fluid 330 can wash away the biopsy samples 320(1), 320(2), and 320(3) from the needle 316, and discharge the biopsy samples 320(1), 320(2), and 320(3) out of the container 304 through the outlet 334. Extra wash fluid 330 can be discharged from the container 304 after the biopsy samples 320(1), 320(2), and 320(3) are flushed off.

Next, the needle 316 of the biopsy device 306 can be re-sheathed into the sheath 314. The biopsy device 306 can be removed from the container 304. In some examples, the container 304 and/or the biopsy device 306 can be reusable. Other operations can be performed on the container 304 and/or the biopsy device 306, such as cleaning, rinsing, disinfecting, sterilizing, steaming, drying, or the like, to make the container 304 and/or the biopsy device 306 reusable. Additionally or alternatively, the container 304 and/or the biopsy device 306 can be disposable.

In some instances, the system 300 for imaging biopsy samples can further include a waste receptacle (not shown), one or more pumps (not shown), and one or more valves (not shown). The waste receptacle is configured to collect and contain the wash fluid. The one or more pumps are configured to move the tissue stain from the container into the waste receptacle.

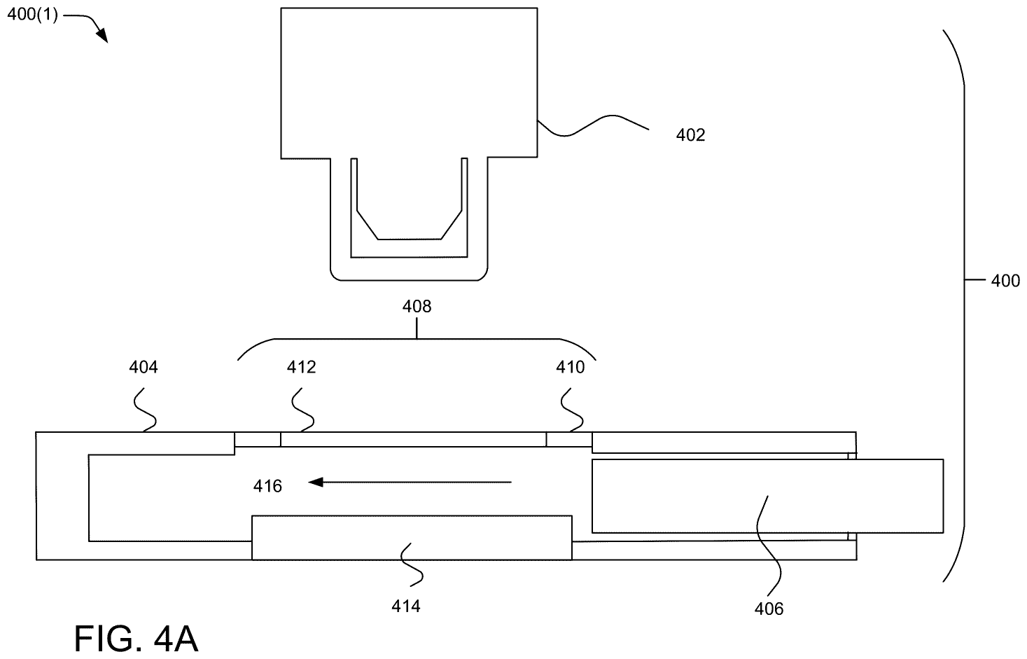

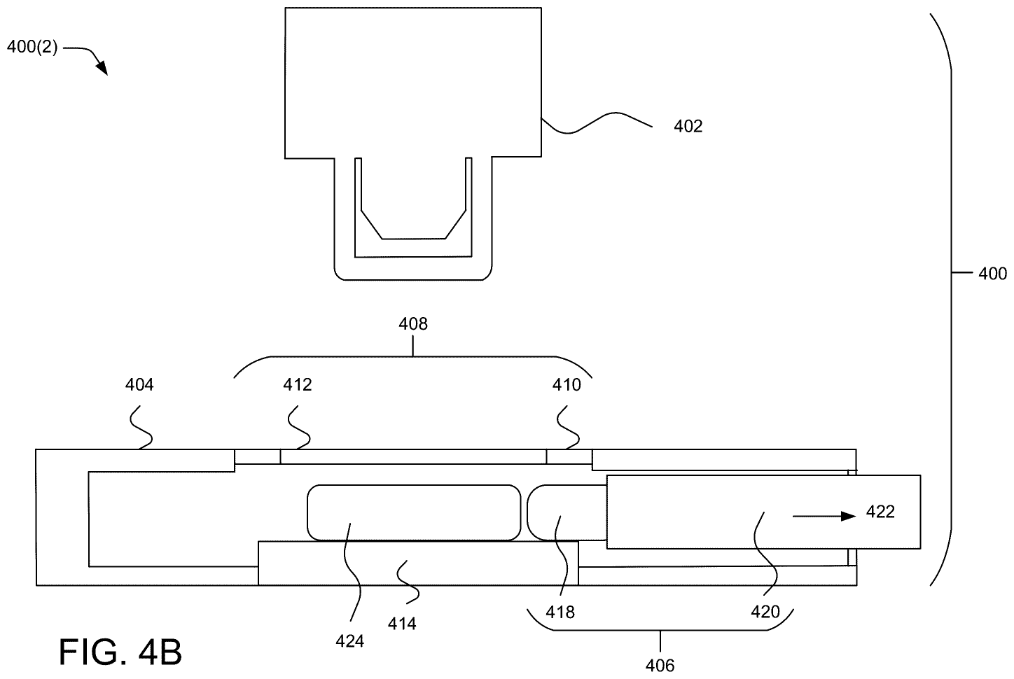

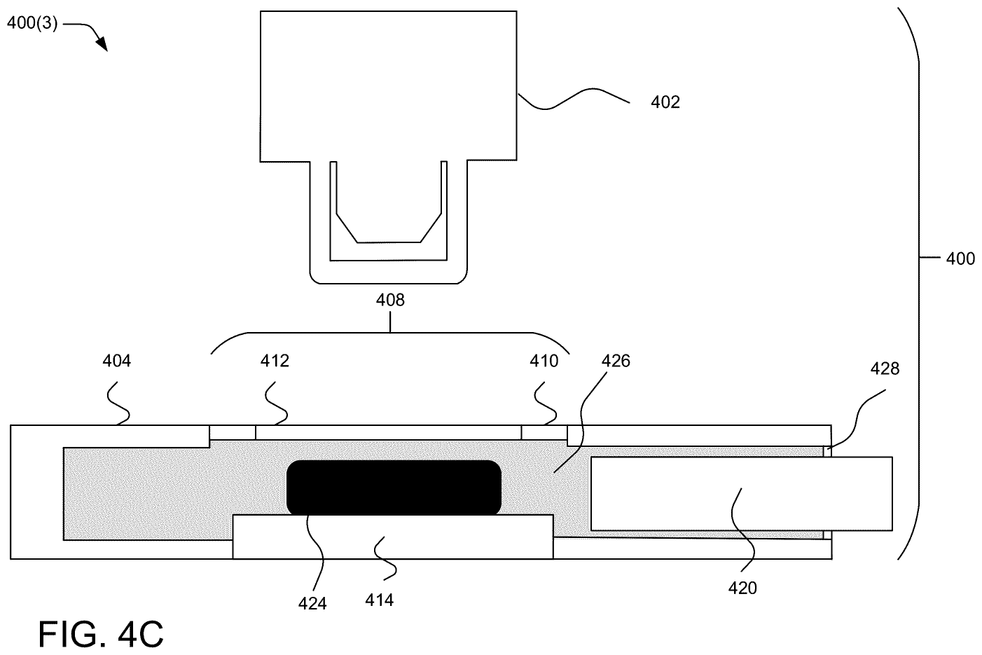

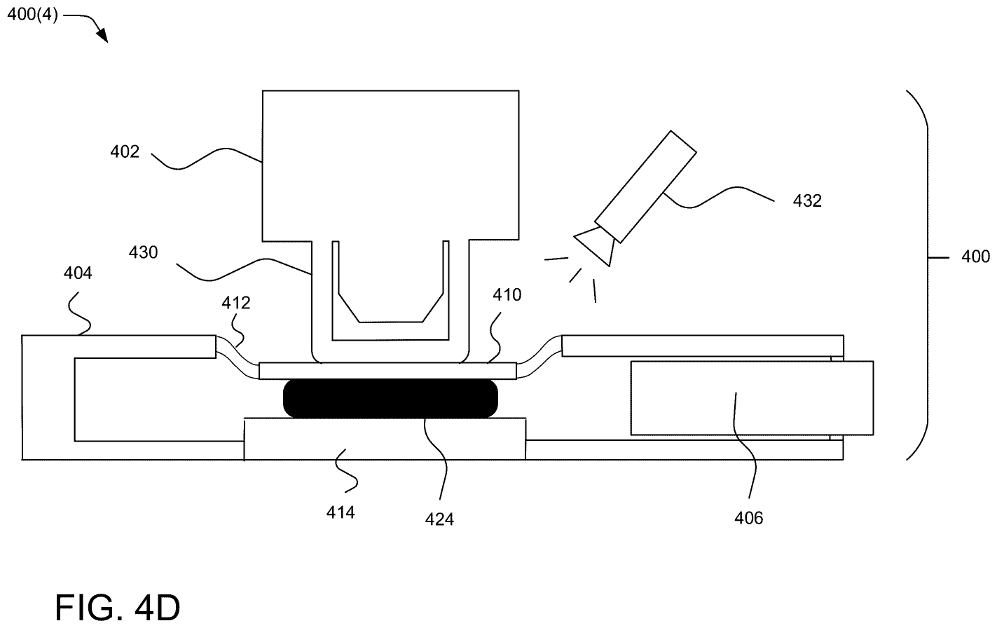

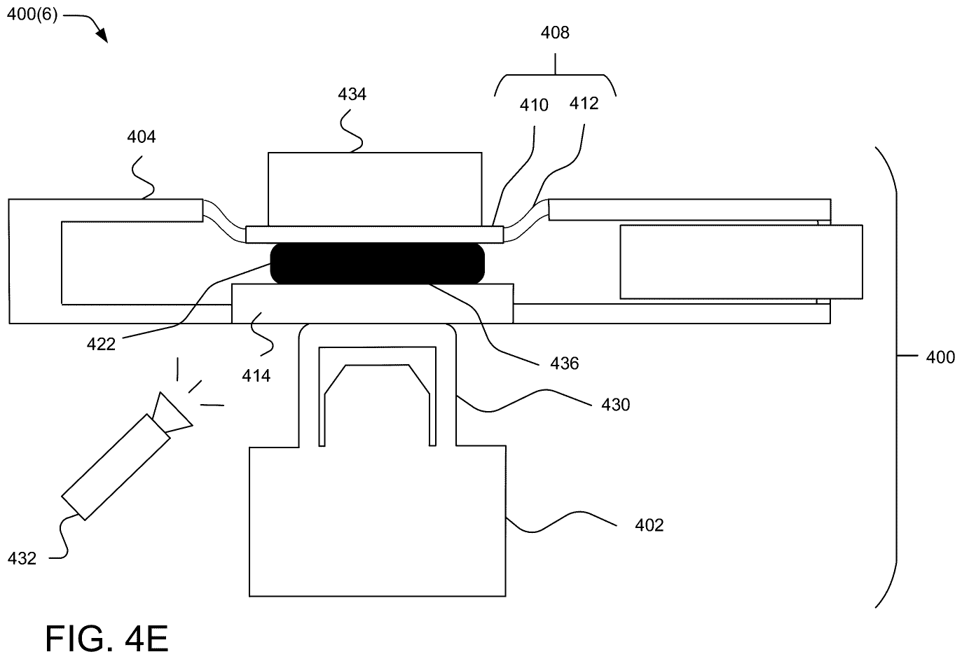

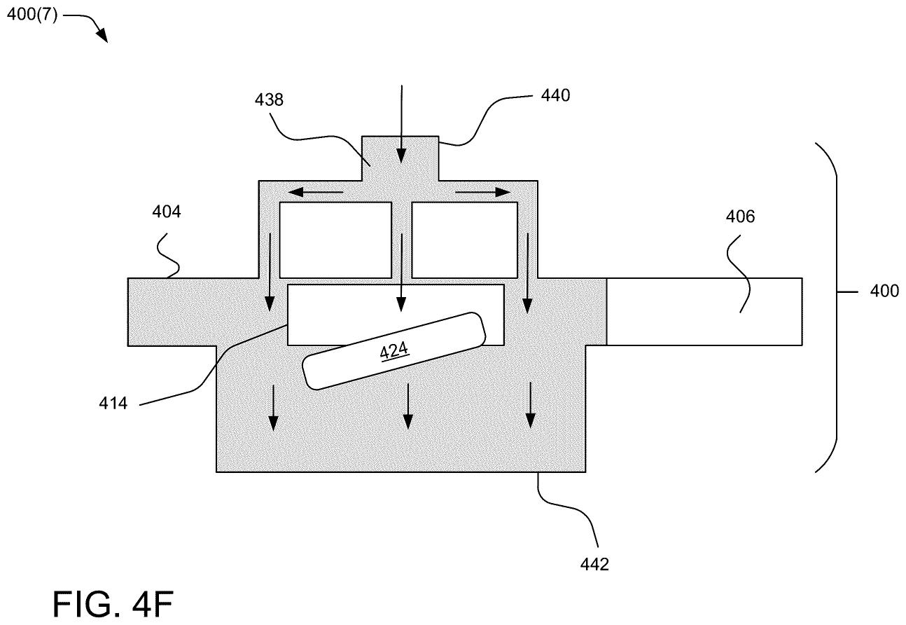

FIG. 4A, FIG. 4B, FIG. 4C, FIG. 4D, FIG. 4E, and 4F illustrate another example procedure for imaging biopsy samples using a system 400 for imaging biopsy samples in accordance with implementations of this disclosure. FIG. 4A illustrates a side view of a system 400 for imaging biopsy samples in accordance with implementations of this disclosure, where a biopsy device carrying biopsy sample(s) is placed into a container of the system 400. Referring to FIG. 4A, at operation 400(1), the system 400 for imaging biopsy samples includes an imaging device 402 and a container 404. In some examples, the imaging device 402 can include, but is not limited to, an optical microscope, an electron microscope, a stereomicroscope, a fluorescence microscope, or the like.

The container 404 can receive a biopsy device 406 which may contain one or more biopsy samples. The container 404 can include a first window 408 which can include a flexible component 410 and a transparent component 412. An outer edge of the flexible component 410 can be coupled to an inner edge of the first window 408. For example, the outer edge of the flexible component 410 can be adhered to the inner edge of the first window 408 using an adhesive. An outer edge of the transparent component 412 can be coupled to an inner edge of the flexible component 410.

The flexible component 410 can be made of elastic material. In some examples, the flexible component 410 can have a ring shape, a bangle shape, a frame shape, a folded bellow shape, or any other shapes that can accommodate the transparent component 412. In some examples, the flexible component 410 can be watertight. The flexible component 410 can be configured to accommodate the transparent component 412 inside.

The transparent component 412 can be made of an inflexible and optically clear material. In some examples, the transparent component 412 can be made of materials that allow light to travel therethrough. Additionally or alternatively, the transparent component 412 can be made of materials that allow electron beam, infrared, ultraviolet, electromagnetic wave, fluorescence, or the like to travel therethrough. Though FIG. 4A shows that the transparent component 412 has a rectangular shape, the transparent component 412 can have other shapes, such as a round shape, a polygon shape, an oval shape, an irregular shape, or the like. The size of the transparent component 412 can be configured to expose the entire biopsy sample. In this example, the biopsy device 406 can be an end-cut needle. The end-cut needle can be configured to collect and preserve biopsy sample(s) from a patient or a recipient. In some examples, a recipient can be an animal, a human, a plant, a living object, or the like. In this example, the biopsy device (the end-cut needle) 406 can be introduced into the container 404 along the direction 416.

The container 404 can further include a second window 414. The second window 414 can be made of an inflexible and optically clear material. In some examples, the second window 414 can be made of materials that allow light to travel therethrough. Additionally or alternatively, the second window 414 can be made of materials that allow electron beam, infrared, ultraviolet, electromagnetic wave, fluorescence, or the like to travel therethrough. In some examples, the second window 414 can have various shapes, such as a rectangular shape, a round shape, a polygon shape, an oval shape, an irregular shape, or the like. The size of the second window 414 can be configured to expose the entire biopsy sample.

FIG. 4B illustrates a side view of the system 400 for imaging biopsy samples in accordance with implementations of this disclosure, where a sheath of the biopsy device is retrieved and the biopsy sample(s) is exposed. Referring to FIG. 4B, at operation 400 (2), the biopsy device 406 can include an end-cut needle 418 and a sheath 420. The sheath 420 of the biopsy device 406 can be retrieved along the direction 422. The needle 418 of the biopsy device 406 can contain one or more biopsy sample(s) 424. The needle 418 can place the biopsy sample(s) 424 under the first window 408 which is suitable for exposing the biopsy sample(s) 424 to the imaging device 402. In some instances, the imaging device 402 can be configured to capture images of the biopsy sample(s) 424 in the container 404 via the first window 408.

FIG. 4C illustrates a side view of the system 400 for imaging biopsy samples in accordance with implementations of this disclosure, where a sheath of the biopsy device is retrieved, and the biopsy sample(s) is exposed. Referring to FIG. 4C, a staining solution 426 can be introduced into the container 404 to stain the biopsy sample(s) 424. The staining solution 426 may contain one or more tissue stains or dyes that enhance contrast for morphological imaging and molecular imaging. After the biopsy sample(s) 424 is stained, the staining solution 426 can be discharged from the container 404 via the one or more ports (not shown). In some examples, the staining solution 426 can stay in the container 204 for a period suitable to stain the biopsy sample(s). For example, various types of stains or dyes in the staining solution can be useful to diagnose different types of pathologies, such as different types of cancers.

In some examples, the system 400 for imaging biopsy samples can further include a staining mechanism (not shown) configured to provide the staining solution 426 to the container 404 via one or more ports (not shown). In some examples, the staining solution 426 can be introduced into the container at a given fluidic rate. In some examples, the one or more ports can include exit ports and entrance ports, where the exit ports are configured to discharge the staining solution 426 out of the container 404, and the entrance ports are configured to introduce the staining solution 426 into the container 404. Additionally or alternatively, the one or more ports can be used for both introducing and discharging the staining solution into/from the container 404. In some examples, a respective port can have a valve configured to open, close, and/or control the flow rate of the staining solution 426.

The container 404 can further include a sealing component 428 configured to seal the container 404 such that the container 404 is fluid-tight. The sealing component 428 can be configured to accommodate the biopsy device 406 when the biopsy device 406 is introduced into the container 404. The sealing component 428 that accepts the biopsy device 406 can be a septum or adjustable component that can be clamped to provide a fluid-tight seal while still allowing the sheath 420 to move along the direction 422. In some examples, the sealing component 428 can be made of silicone, rubber, polytetrafluoroethylene (PTFE), fluorosilicone (FVMQ), polyurethane, or the like.

In some examples, a fixation reagent can be introduced into the container 404 to fix the biopsy sample(s) 424. Examples of fixation reagents include but are not limited to ethanol, formalin, acetone, picric acid, wax, or the like.

FIG. 4D illustrates a side view of the system 400 for imaging biopsy samples in accordance with implementations of this disclosure, where the compressing component applies a pressing force on the first window of the container to compress the biopsy sample(s). Referring to FIG. 4D, at operation 400 (4), after the staining solution 426 is discharged from the container 404, the system 400 for imaging biopsy samples can control the imaging device 402 to press, via the support assembly 430, against the transparent component 412 of the window so as to compress the biopsy sample(s) 424. In this example, in response to the pressing force applied by the imaging device 402 via the support assembly 430, the flexible component can deform, and the transparent component 412 can be displaced to contact the biopsy sample(s) 424 to compress the biopsy sample(s) 424.

The system 400 for imaging biopsy samples can control the imaging device 402 to capture images of the biopsy sample(s) 424 in the container 404 via the transparent component 412. In some examples, the imaging device 202 can have a focus plane on the surface of the transparent component 412. The focus plane can be adjusted as needed. In some examples, the imaging device 402 can include a wide FOV objective lens. In some examples, the wide FOV objective lens can be used for a low-magnification wide FOV imaging. Additionally or alternatively, a high-resolution objective lens can be used, while the FOV thereof can be much smaller than the wide FOV objective lens. In some examples, the imaging device 402 can move along various directions (e.g., horizontally, vertically, laterally, or the like) while applying the pressing force to the surface of the transparent component 412. The imaging device 402 can capture images of different parts of the biopsy sample(s) 424 while moving along various directions.

After taking the images of the biopsy sample(s) 424, the imaging device 402 can be lifted from the transparent component 412 of the window. As the force applied to the transparent component 412 goes away, the transparent component 412 can go back to the original position, and the flexible component 410 can recover to the original shape and position.

In some examples, the system 400 for imaging biopsy samples can also include a light source 432 configured to emit light through the transparent component 412. Examples of the light source 432 can include, but are not limited to, a flashlight, lantern, limelight, laser device, light-emitting diode (LED), epi-illumination device, excitation light device, or the like.

FIG. 4E illustrates a side view of the system 400 for imaging biopsy samples in accordance with implementations of this disclosure, where the compressing component applies a pressing force on the first window of the container, and the imaging device applies a force on the second window of the container to compress the biopsy sample(s). Referring to FIG. 4E, at operation 400(6), additionally or alternatively, the imaging device 402 can be arranged proximate to the second window 414 such that the imaging device 402 can capture images of the biopsy sample(s) 424. In this example, the system 400 for imaging biopsy samples can further include a pressing component 434, arranged proximate to the first window. The pressing component 434 can be configured to apply a force against the first window 408. In this example, the system 400 for imaging biopsy can control the pressing component 434 to press the transparent component 412 of the first window 408, such that the imaging device 402 can press, via the support assembly 430, the second window 414. As such, the biopsy sample(s) 424 can be compressed by the transparent component 412 of the first window 408 and the second window 414. In this example, in response to the pressing force applied by the pressing component 434, the flexible component 410 can deform, and the transparent component 412 can be displaced to press the biopsy sample(s) 424. Other the other hand, the imaging device 402 can press, via the support assembly 430, the second window 414 and apply a force against the second window 414, such that the biopsy sample(s) 424 can be squeezed between the first window 408 and the second window 414.

The system 400 for imaging biopsy samples can control the imaging device 402 to capture images of the biopsy sample(s) 424 in the container 404 via the second window 414. In some examples, the imaging device 402 can have a focus plane on the surface of the second window 414 or the surface 436 of the biopsy sample(s) 424. The focus plane can be adjusted as needed. In some examples, the imaging device 402 can move along various directions (e.g., horizontally, vertically, laterally, or the like) while applying the pressing force to the surface of the second window 414. The imaging device 402 can capture images of different parts of the biopsy sample(s) 424 while moving along various directions.

After taking the images of the biopsy sample(s) 424, the pressing component 434 can be lifted from the surface of the first window 408, and the imaging device 402 can be taken away from the surface of the second window 414. As the pressing force applied to the transparent component 412 goes away, the transparent component 412 can go back to the original position, and the flexible component 410 can recover to the original shape and position.

In some examples, the system 400 for imaging biopsy samples can also include a light source 432 configured to emit light through the transparent component 412. Examples of the light source 432 can include, but are not limited to, a flashlight, lantern, limelight, laser device, light-emitting diode (LED), epi-illumination device, excitation light device, or the like.

FIG. 4F illustrates a top view of the system for imaging biopsy samples in accordance with implementations of this disclosure, where the biopsy sample(s) is flushed out. Referring to FIG. 4F, at operation 400(7) the system 400 for imaging biopsy samples can introduce wash fluid 438 into the container 404 to flush the container 404 to discharge the biopsy sample(s) 424. In some examples, the wash fluid 438 can be introduced into the container 404 via an inlet 440. The wash fluid 438 can be introduced into the container 404 at a given fluidic rate. Then, the wash fluid 438 can wash away the biopsy sample(s) 424, and discharge the biopsy sample(s) 424 out of the container 404 through the outlet 442. The wash fluid 438 can be discharged from the container 404 after the biopsy samples 424 are flushed off.

Next, biopsy device 406 can be removed from the container 404. In some examples, the container 404 and/or the biopsy device 406 can be reusable. Other operations can be performed on the container 404 and/or the biopsy device 406, such as cleaning, rinsing, disinfecting, sterilizing, steaming, or the like, to make sure that the container 404 and/or the biopsy device 406 are reusable. Additionally or alternatively, the container 404 and/or the biopsy device 406 can be disposable.

In some instances, the system 400 for imaging biopsy samples can further include a waste receptacle (not shown) and one or more pumps (not shown). The waste receptacle is configured to collect and contain the wash fluid 438. The one or more pumps are configured to move the tissue stain from the container into the waste receptacle.

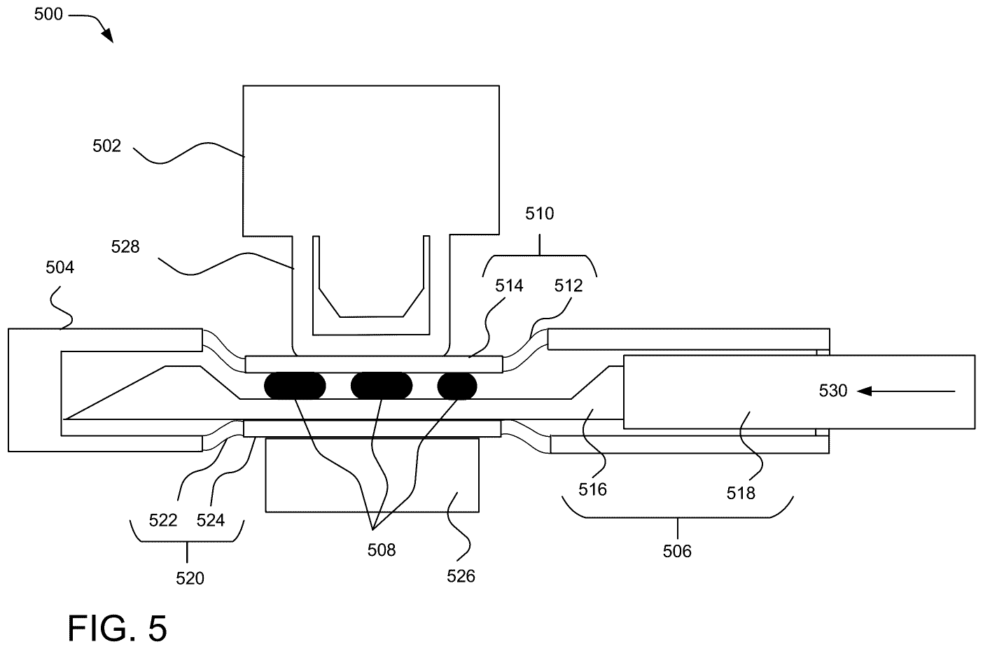

FIG. 5 illustrates a side view of an example biopsy imaging system 500 in accordance with implementations of this disclosure, where a container of the biopsy imaging system 500 includes two windows. Referring to FIG. 5, biopsy imaging system 500 includes an imaging device 502 and a container 504. In some examples, the imaging device 502 can include, but is not limited to, an optical microscope, an electron microscope, a stereomicroscope, a fluorescence microscope, or the like.

The container 504 can receive a biopsy device 506 which may contain one or more biopsy samples 508. The container 504 can include a first window 510 which includes a first flexible component 512 and a first transparent component 514. An outer edge of the first flexible component 512 can be coupled to an inner edge of the first window 510. For example, the outer edge of the first flexible component 512 can be adhered to the inner edge of the first window 510 using an adhesive. An outer edge of the first transparent component 514 can be coupled to an inner edge of the first flexible component 512.

The first flexible component 512 can be made of elastic material. In some examples, the first flexible component 512 can have a ring shape, a bangle shape, a frame shape, a folded bellow shape, or any other shapes that can accommodate the first transparent component 514. In some examples, the first flexible component 512 can be watertight. The first flexible component 512 can be configured to accommodate the first transparent component 514 inside.

The first transparent component 514 can be made of an inflexible and optically clear material. In some examples, the first transparent component 514 can be made of materials that allow light to travel therethrough. Additionally or alternatively, the first transparent component 514 can be made of materials that allow electron beam, infrared, ultraviolet, electromagnetic wave, fluorescence, or the like to travel therethrough. The first transparent component 514 can have various shapes, such as a rectangular shape, a round shape, a polygon shape, an oval shape, an irregular shape, or the like. The size of the first transparent component 514 can be configured to expose the entire biopsy sample(s) 508.

The biopsy device 506 includes a needle 516 and a sheath 518. In this example, the needle 516 is a side-curt needle configured to collect and preserve biopsy sample(s) from a patient or a recipient. In some examples, a recipient can be an animal, a human, a plant, a living object, or the like. In this example, the biopsy device 506 can be introduced into the container 504 along the direction 530.

The container 504 can further include a second window 520, which includes a second flexible component 522 and a second transparent component 524. An outer edge of the second flexible component 522 can be coupled to an inner edge of the second window 520. For example, the outer edge of the second flexible component 522 can be adhered to the inner edge of the second window 520 using an adhesive. An outer edge of the second transparent component 524 can be coupled to an inner edge of the second flexible component 522. The second flexible component 522 and the second transparent component 524 can be implemented in the same way as the first flexible component 512 and the first transparent component 514. Details are not repeated here.

The biopsy imaging system 500 can further include a pressing component 526 configured to apply a pressing force against the second transparent component 524 of the second window 520. In this example, the imaging device 502 can apply a pressing force to the window 510 via a support assembly 528, and capture images of the biopsy sample(s) 508 in the same way as the imaging device 302 described with respect to FIG. 3A to FIG. 3F. The pressing component 526 can apply a pressing force to the second window 520 in the same way as the pressing component 526 described with respect to FIG. 4E. For example, the imaging device 502 can apply a pressing force to the window 510 via a support assembly 528, and the pressing component 526 can apply a pressing force to the second window 520. In this example, in response to the pressing force applied by the imaging device 502 via the support assembly 528, the first flexible component 512 can deform, and the first transparent component 514 can be displaced to contact the biopsy sample(s) 508. On the other hand, in response to the pressing force applied by the pressing component 526, the second flexible component 522 can deform, and the second transparent component 524 can be displaced to compress the biopsy sample(s) 508. In this example, the needle 516 is still inside the container 504 when the biopsy sample(s) 508 is compressed. Additionally or alternatively, the needle 516 can be removed before the biopsy sample(s) 508 is compressed, and the biopsy sample(s) 508 can contact the second transparent component 524. Next, the imaging device 502 can capture images of the biopsy sample(s) 508 in the same way as the imaging device 302 described with respect to FIG. 3A to FIG. 3F.

Additionally or alternatively, the position of the imaging device 502 and the position of the pressing component 526 can be exchanged. For example, the imaging device 302 can be arranged proximate to the second window 520, while the pressing component 526 can be arranged proximate to the first window 510. In such an example, the imaging device 502 and the pressing component 526 can work in the same way as described above, just the positions of the imaging device 502 and the pressing component 526 are swapped.

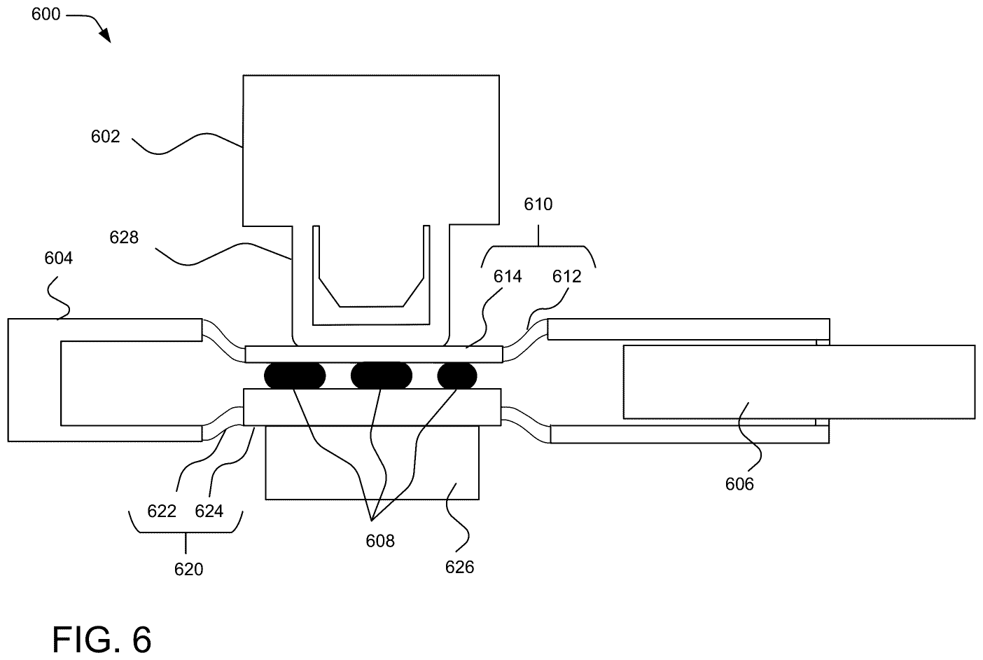

FIG. 6 illustrates a side view of an example biopsy imaging system 600 in accordance with implementations of this disclosure, where a container of the biopsy imaging system 600 includes two windows, and the needle of the biopsy device is retrieved. Referring to FIG. 6, biopsy imaging system 600 includes an imaging device 602 and a container 604. In some examples, the imaging device 602 can include, but is not limited to, an optical microscope, an electron microscope, a stereomicroscope, a fluorescence microscope, or the like.

The container 604 can receive a biopsy device 606 which may contain one or more biopsy samples 608. The container 604 can include a first window 610 which includes a first flexible component 612 and a first transparent component 614. An outer edge of the first flexible component 612 can be coupled to an inner edge of the opening of the first window 610. For example, the outer edge of the first flexible component 612 can be adhered to the inner edge of the first window 610 using an adhesive. An outer edge of the first transparent component 614 can be coupled to an inner edge of the first flexible component 612.

The first flexible component 612 can be made of elastic material. In some examples, the first flexible component 612 can have a ring shape, a bangle shape, a frame shape, a folded bellow shape, or any other shapes that can accommodate the first transparent component 614. In some examples, the first flexible component 612 can be watertight. The first flexible component 612 can be configured to accommodate the first transparent component 614 inside.

The first transparent component 614 can be made of an inflexible and optically clear material. In some examples, the first transparent component 614 can be made of materials that allow light to travel therethrough. Additionally or alternatively, the first transparent component 614 can be made of materials that allow electron beam, infrared, ultraviolet, electromagnetic wave, fluorescence, or the like to travel therethrough. The first transparent component 614 can have various shapes, such as a rectangular shape, a round shape, a polygon shape, an oval shape, an irregular shape, or the like. The size of the first transparent component 614 can be configured to expose the entire biopsy sample(s) 608.

In this example, the biopsy device 606 can be introduced into the container 604 and placed the biopsy sample(s) 608 in the container 604. For example, the biopsy sample(s) 608 can be placed between the first window 610 and the second window 620 such that the biopsy sample(s) 608 is exposed to the first window 610 and the second window 620.

The container 604 can further include a second window 620, which includes a second flexible component 622 and a second transparent component 624. An outer edge of the second flexible component 622 can be coupled to an inner edge of the opening of the second window 620. For example, the outer edge of the second flexible component 622 can be adhered to the inner edge of the second window 620 using an adhesive. An outer edge of the second transparent component 624 can be coupled to an inner edge of the second flexible component 622. The second flexible component 622 and the second transparent component 624 can be implemented in the same way as the first flexible component 612 and the first transparent component 614. Details are not repeated here.

The biopsy imaging system 600 can further include a pressing component 626 configured to apply a pressing force against the second transparent component 624 of the second window 620. In this example, the imaging device 602 can apply a pressing force to the window 610 via a support assembly 628, and capture images of the biopsy sample(s) 608 in the same way as the imaging device 302 described with respect to FIG. 3A to FIG. 3F. The pressing force can be implemented mechanically (e.g., by a motor, actuator, etc.) or manually (e.g., by a manually operated handle or press, etc.). The pressing component 626 can apply a pressing force to the second window 620 in the same way as the pressing component 626 described with respect to FIG. 4E. For example, the imaging device 602 can apply a pressing force to the window 610 via a support assembly 628, and the pressing component 626 can apply a pressing force to the second window 620. In this example, in response to the pressing force applied by the imaging device 602 via the support assembly 628, the first flexible component 612 can deform, and the first transparent component 614 can be displaced to contact the biopsy sample(s) 608. On the other hand, in response to the pressing force applied by the pressing component 626, the second flexible component 622 can deform, and the second transparent component 624 can be displaced to compress the biopsy sample(s) 608. In this example, unlike the example described with respect to FIG. 5, the needle of the biopsy device 606 is retrieved, and the biopsy sample(s) 608 is placed on top of the second window 620 of the container 604 when the biopsy sample(s) 608 is compressed. Next, the imaging device 602 can capture images of the biopsy sample(s) 608 in the same way as the imaging device 302 described with respect to FIG. 3A to FIG. 3F.

Additionally or alternatively, the position of the imaging device 602 and the position of the pressing component 626 can be exchanged. For example, the imaging device 302 can be arranged proximate to the second window 620, while the pressing component 626 can be arranged proximate to the first window 610. In such an example, the imaging device 602 and the pressing component 626 can work in the same way as described above, just the positions of the imaging device 602 and the pressing component 626 are swapped.

In some instances, the sidewalls and the back side (opposite to the window) of the container (e.g., the container 404 in FIG. 4A to FIG. 4F, the container 504 in FIG. 5, and the container 604 in FIG. 6) can be made of elastic material. As described above, the imaging device (e.g., the imaging device 402 in FIG. 4A to FIG. 4F, the imaging device 502 in FIG. 5, and imaging device 602 in FIG. 6) can apply a pressing force, via a support assembly, against the window of the container. Additionally or alternatively, there can be a pressing component (e.g., the pressing component 526 in FIG. 5 and the pressing component 626 in FIG. 6) configured to apply a pressing force to the backside of the container. In this example, the sidewalls and the backside of the container that are made of elastic material can deform in response to the pressing force applied by the imaging device and/or the pressing component. As such, the biopsy sample(s) (e.g., the biopsy sample(s) in FIG. 4A to FIG. 4F, the biopsy sample(s) in 508 in FIG. 5, and the biopsy sample(s) 608 in FIG. 6) can be compressed. Then, the imaging device can capture images of the compressed biopsy sample(s).

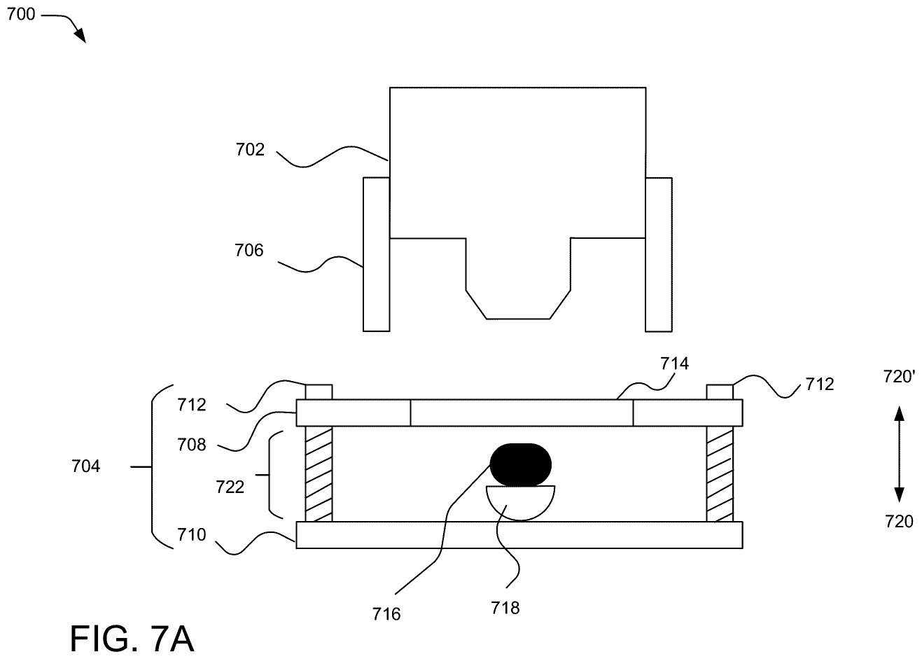

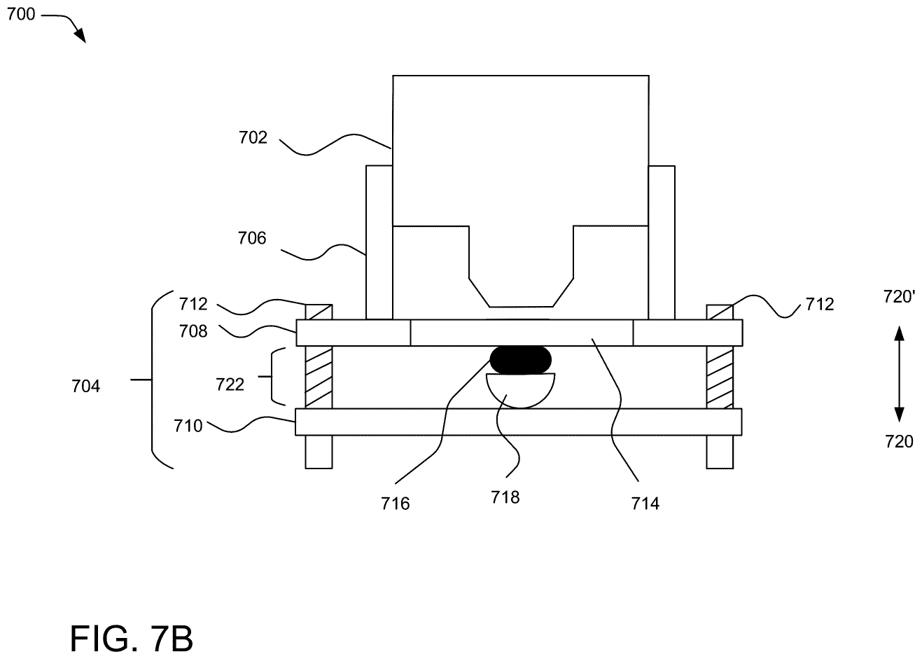

FIG. 7A and FIG. 7B illustrate side views of another example biopsy imaging system 700 in accordance with implementations of this disclosure. Referring to FIG. 7A, the biopsy imaging system 700 includes an imaging device 702 and a compressing mechanism 704. The imaging device 702 can include a support assembly 706 configured to apply pressing forces to the compressing mechanism 704 by the outer housing that contacts platform 708. The imaging device 702 can be implemented in the same way as the imaging device 202 described with respect to FIG. 2A and FIG. 2B, the imaging device 302 described with respect to FIG. 3A to FIG. 3E, the imaging device 402 described with respect to FIG. 4A and FIG. 4B, the imaging device 502 described with respect to FIG. 5, or the imaging device 602 described with respect to FIG. 6.

In this example, the compressing mechanism 704 includes a platform 708, a base 710, and one or more lifting component(s) 712. The platform 708 is configured to be subject to the pressing forces received by the imaging device 702 via the support assembly 706, for example, when the compressing mechanism 704 moves vertically along the directions 720 and 720′. The platform 708 includes a window 714 which is suitable for exposing the biopsy sample(s) 716 to the imaging device 702. In some instances, the window 714 can be made of an inflexible and optically clear material.

The base 710 is configured to receive and accommodate a biopsy device 718. The biopsy device 718 can be implemented in a similar way to the biopsy device 108 described with respect to FIG. 1, the biopsy device 206 described with respect to FIG. 2A and FIG. 2B, the biopsy device 306 described with respect to FIG. 3A to FIG. 3F, the biopsy device 406 described with respect to FIG. 4A to FIG. 4F, the biopsy device 506 described with respect to FIG. 5, or the biopsy device 606 described with respect to FIG. 6.

The one or more lifting component(s) 712 can be coupled to the platform 708 and the base 710. The one or more lifting component(s) 712 can be configured to allow the platform 708 and the base 710 to move along the lifting component(s) 712 with respect to each other.

Referring to FIG. 7B, for example, the compressing mechanism 704 can move along the direction 720′ such that the platform 708 contacts the support assembly 706 of the imaging device 702. While the imaging device 702 receives, via the support assembly 706, a pressing force applied by the window 714 of the platform 708, the base 710 can move along the one or more lifting component(s) 712 in the direction 720′. That is, the base 710 is configured to press the biopsy sample(s) 716 into the window 714. As such, the window 714 of the platform 708 can contact the biopsy sample(s) 716 which can be compressed and flattened against the window 714 as the base 710 moves vertically within the compressing mechanism 704. Though FIG. 7 shows the biopsy device 718 on the base 710, in some examples, the biopsy device 718 can be retrieved before the biopsy sample(s) is compressed between the platform 708 and the base 710. In some instances, a respective lifting component 712 can include screw threads 722 to facilitate the movement of the base 710 along the lifting component 712. In some instances, the base 710 can be lifted vertically along the direction 720′ towards the platform 708 so as to compress the biopsy sample(s) 716. As such, a spacing between the platform 708 and the base 710 can be reduced. Additionally, one or more smooth slider poles with spring assembly can be arranged between the platform 708 and the base 710 to allow both the base 710 and window 714 to remain parallel during the compression of the biopsy sample(s) 716.

The biopsy imaging system 700 can control the imaging device 702 to capture images of the biopsy sample(s) 716 via the window 714. In some examples, the imaging device 702 can have a focus plane on the surface of the window 714 or the surface of the biopsy sample(s) 716. The focus plane can be adjusted as needed. In some examples, the imaging device 702 can move along various directions (e.g., horizontally, vertically, laterally, or the like) while applying the pressing force to the window 714. The imaging device 702 can capture images of different parts of the biopsy sample(s) 716 while scanning the window 714.

After taking the images of the biopsy sample(s) 716, the compressing mechanism 704 can be lowered along the direction 720 to move away from the imaging device 702. The base 710 can move back to the original position or a position where the platform 708 does not compress the biopsy sample(s) 716. The biopsy sample(s) 716 can be removed from biopsy imaging system 700. In some instances, other operations can be performed on the biopsy imaging system 700, such as cleaning, rinsing, disinfecting, sterilizing, steaming, drying, or the like, to make the biopsy imaging system 700 reusable.

Although an example where the base 710 is lifted towards the platform 708 is described above, in some examples, it should be understood that the platform 708 can move downwards along the direction 720 to compress the biopsy sample(s) 716. The imaging device 702 can apply a pressing force against the window 714 of the platform 708. The biopsy imaging system 700 can control the imaging device to capture images of the biopsy sample(s) 716. Then, the imaging device 702 and the compressing mechanism 704 can be taken apart from each other, either by moving the imaging device 702 upwards along the direction 720′ or by moving the compressing mechanism 704 downwards along the direction 720, such that the imaging device 702 does not contact the window 714 of the platform. Then, the platform 708 can be lifted from the same biopsy sample(s) 716 such that the platform 708 does not compress the biopsy sample(s) 716. The biopsy imaging system 700 can be rinsed, disinfected, dried, or the like to be reused for imaging an additional sample.

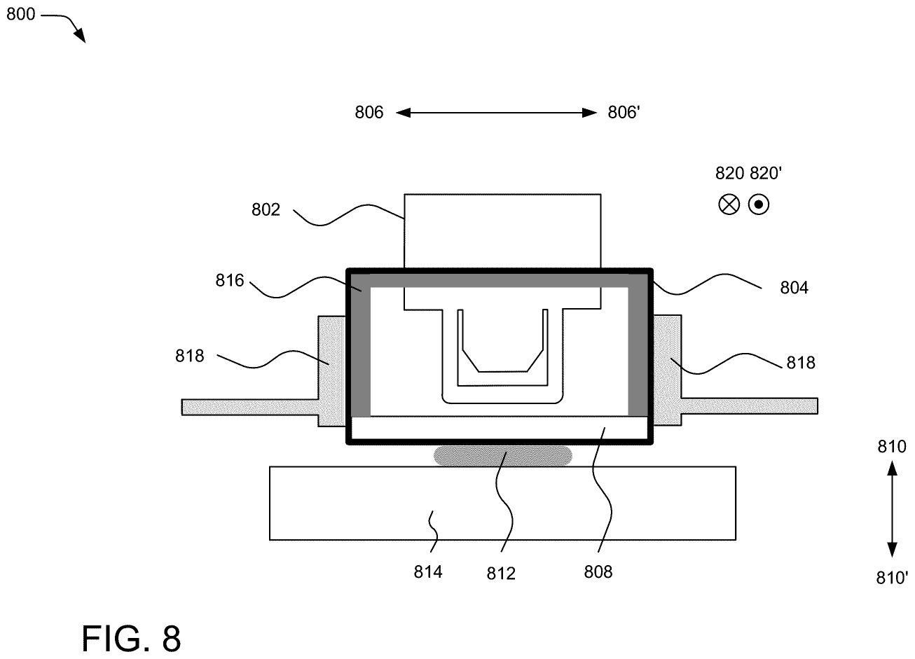

FIG. 8 illustrates a side view of another example biopsy imaging system 800 in accordance with implementations of this disclosure. Referring to FIG. 8, the biopsy imaging system 800 includes an imaging device 802 and a piston-stage 804. The piston-stage 804 can be arranged to accommodate and support the imaging device 802 and allow the imaging device 802 to move within the piston-stage 804 along the direction 806 or the direction 806′. The imaging device 802 can be implemented in the same way as the imaging device 202 described with respect to FIG. 2A and FIG. 2B, the imaging device 302 described with respect to FIG. 3A to FIG. 3E, the imaging device 402 described with respect to FIG. 4A and FIG. 4B, the imaging device 502 described with respect to FIG. 5, the imaging device 602 described with respect to FIG. 6, or the imaging device 702 described with respect to FIG. 7A and FIG. 7B.

The piston-stage 804 can include a window 808. The window 808 can be made of an inflexible and optically clear material. In some examples, the window 808 can be made of materials that allow light to travel therethrough. Additionally or alternatively, the transparent component 220 can be made of materials that allow electron beam, infrared, ultraviolet, electromagnetic wave, fluorescence, or the like to travel therethrough.

The piston-stage 804 can move along the direction 820 (perpendicular to the surface of the drawing, pointing inwards) or the direction 820′ (perpendicular to the surface of the drawing, pointing outwards). In some instances, biopsy imaging system 800 can further include a base plate 814 configured to support the biopsy sample(s) 812. The base plate 814 can move vertically along the direction 810 and the direction 810′. For example, when the base plate 814 moves upwards along the direction 810, the window 808 can contact the biopsy sample(s) 812, and the biopsy sample(s) 812 can be compressed between the window 808 and the base plate 814.

The biopsy imaging system 800 can control the imaging device 802 to capture images of the biopsy sample(s) 812 via the window 808. In some examples, the imaging device 802 can have a focus plane on the surface of the window 808 or the surface of the biopsy sample(s) 812. The focus plane can be adjusted as needed. The imaging device 802 can capture images of different parts of the biopsy sample(s) 812 while scanning the window 808.

After taking the images of the biopsy sample(s) 812, the base plate 814 can move along the direction 810′ such that the piston-stage 804 and the base plate 814 can be apart from each other. For example, the base plate 814 can move back to the original position or a position where the window 808 does not compress the biopsy sample(s) 812. The biopsy sample(s) 812 can be removed from biopsy imaging system 800.

In some instances, the piston-stage 804 can further include sliding seal 816 such as a rubber gasket configured to ensure the piston-stage 804 is fluid-tight. In some instances, the piston-stage 804 can further include one or more support components 818 configured to support the piston-stage 804.

In some instances, the piston-stage 804 can be filled with a fluid such as a staining solution, a wash fluid, or the like. The staining solution may contain one or more tissue stains or dyes that enhance contrast for morphological imaging and molecular imaging. In some instances, the movement of the piston-stage 804 can also act as a pump to express, fill, or agitate the fluid inside the piston-stage 804.

In some instances, other operations can be performed on the biopsy imaging system 800, such as cleaning, rinsing, disinfecting, sterilizing, steaming, drying, or the like, to make the biopsy imaging system 800 reusable.

FIG. 9A and FIG. 9B illustrate an example biopsy imaging system 900 in accordance with implementations of this disclosure. Referring to FIG. 9A, the biopsy imaging system 900 includes an imaging device 902, a compression mechanism 904, a staining mechanism 906 (such as a dispenser or the like), and a transportation mechanism 908. The imaging device 902 can be implemented in the same way as the imaging device 202 described with respect to FIG. 2A and FIG. 2B, the imaging device 302 described with respect to FIG. 3A to FIG. 3E, the imaging device 402 described with respect to FIG. 4A and FIG. 4B, the imaging device 502 described with respect to FIG. 5, the imaging device 602 described with respect to FIG. 6, the imaging device 702 described with respect to FIG. 7A and FIG. 7B, or the imaging device 802 described with respect to FIG. 8.

The compression mechanism 904 is configured to receive a pressing force to the biopsy sample(s) 910 to compress and fix the biopsy sample(s) 910. The compression mechanism 904 can be implemented to include the window 112 described with respect to FIG. 1, the window 216 described with respect to FIG. 2A and FIG. 2B, the window 308 described with respect to FIG. 3A to FIG. 3E, the first window 408 and/or the second window 414 described with respect to FIG. 4A to FIG. 4E, the first window 510 and/or the second window 520 described with respect to FIG. 5, the first window 610 and/or the second window 620 described with respect to FIG. 6, the platform 708 described with respect to FIG. 7A and FIG. 7B, or the piston-stage 804 described with respect to FIG. 8.

The staining mechanism 906 is configured to provide a staining solution 912 to the biopsy sample(s) 910 to stain the biopsy sample(s) 910. The staining solution 912 may contain one or more tissue stains or dyes that enhance contrast for morphological imaging and molecular imaging. In some examples, the staining solution 912 may include different types of stains or dyes that are useful for diagnosing different types of pathologies, such as different types of cancers. In some examples, the staining mechanism 906 can include a pump and a dispenser. The pump can be configured to move the staining solution to the dispenser. The dispenser can be configured to dispense the staining solution (such as in droplets) to the biopsy sample(s) 910. The dispenser can be arranged to dribble along the length of the needle 916 of the biopsy device 914.

The transportation mechanism is configured to receive and move a biopsy device 914. In this example, the biopsy device 914 can be a sheathed needle which includes a sheath 918 and a side-cut needle 916. The sheath 918 can be configured to accommodate the side-cut needle 916. The side-cut needle 916 can be configured to collect and preserve biopsy samples 910 from a patient or a recipient. In some examples, a recipient can be an animal, a human, a plant, a living object, or the like.

In this example, after the biopsy sample(s) 910 is stained, the transportation mechanism 908 can be controlled to move along the direction 920, the direction 920′, the direction 924, and the direction 924′ to transfer the biopsy device 914 together with the biopsy sample(s) 910 to a desired position, for example, a position facing the staining mechanism 906, a position facing the compression mechanism 904, or the like.

Referring to FIG. 9B, the transportation mechanism 908 can move upwards towards the compression mechanism 904 along the direction 924, such that the biopsy sample(s) 910 can contact the compression mechanism 904. In some instances, the compression mechanism 904 can move upwards along the direction 924 to contact the imaging device 902. The imaging device 902 can receive a pressing force via a support assembly 924 from the compression mechanism 904 applied by the vertical transportation mechanism 908. As such, the biopsy sample(s) 910 can be compressed and fixed between the compression mechanism 904 and the transportation mechanism 908. The biopsy imaging system 900 can control the imaging device 902 to capture images of the biopsy sample(s) 910 via the compression mechanism 904. In some examples, the imaging device 902 can have a focus plane on the surface of the biopsy sample(s) 910. The focus plane can be adjusted as needed. In some examples, the imaging device 902 can move along various directions (e.g., horizontally, vertically, laterally, or the like). Additionally or alternatively, the transportation mechanism 908 can be controlled to move the biopsy device 914 together with the biopsy sample 910 along various directions (e.g., horizontally, vertically, laterally, or the like) while the imaging device 902 is capturing images of the biopsy sample(s). The imaging device 902 can capture images of different parts of the biopsy sample(s) 910 while moving along various directions.

After taking the images of the biopsy sample(s) 910, the transportation mechanism 908 can move downwards along the direction 924′ such that the biopsy sample(s) 910 can be taken away from the compression mechanism 904. Additionally or alternatively, the imaging device 902 can be lifted from the compression mechanism 904. As the force applied to the compression mechanism 904 goes away, the compression mechanism 904 can go back to the original position or a position that does not compress the biopsy sample(s) 910. Though in this example, the side-cut needle 916 is placed on the transportation mechanism 908 when the biopsy sample(s) 910 is compressed, in some examples, the side-cut needle 916 can be retrieved, and the biopsy sample(s) 910 can be placed on the transportation mechanism 908 directly.

Other operations can be performed on biopsy imaging system 900, such as cleaning, rinsing, disinfecting, sterilizing, steaming, drying, or the like, to make biopsy imaging system 900 reusable.

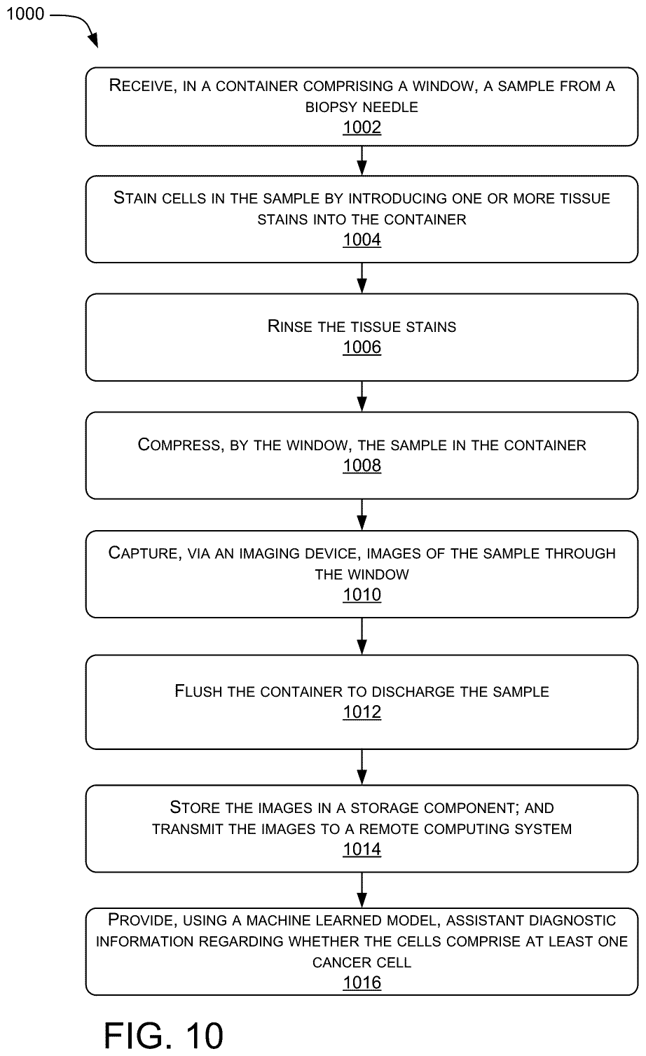

FIG. 10 illustrates a flow chart of an example process 1000 for imaging biopsy samples in accordance with implementations of this disclosure. In some examples, at least some operations of the process 1000 may be performed by one or more systems for imaging biopsy samples described above, such as the system 102 described with respect to FIG. 1, the system 200 described with respect to FIG. 2A and FIG. 2B, the system 300 described with respect to FIG. 3A to FIG. 3F, the system 400 described with respect to FIG. 4A to FIG. 4F, the system 500 described with respect to FIG. 5, the system 600 described with respect to FIG. 6, the system 700 described with respect to FIG. 7A and FIG. 7B, the system 800 described with respect to FIG. 8, or the system 900 described with respect to FIG. 9A and FIG. 9B. Note that the operations of the process 1000 are not limited to the orders presented. Rather, the operations of the process 1000 can be performed in any proper order. Moreover, at least some operations of the process 1000 can be repeated, combined, or split to implement techniques of this disclosure.

At operation 1002, the process can include receiving, in a container including a window, a sample from a biopsy needle. The container can receive a biopsy device that may contain one or more biopsy samples. The window of the container can include a flexible component and a transparent component. An outer edge of the flexible component can be coupled to an inner edge of an opening of the window. For example, the outer edge of the flexible component can adhere to an opening of the inner edge of the window using an adhesive. An outer edge of the transparent component can be coupled to an inner edge of the flexible component.

The flexible component can be made of elastic material. In some examples, the flexible component can have a ring shape, a bangle shape, a frame shape, a folded bellow shape, or any other shapes that can accommodate the transparent component. In some examples, the flexible component can be watertight. The flexible component can be configured to accommodate the transparent component inside.

The transparent component can be made of an inflexible and optically clear material. In some examples, the transparent component can be made of materials that allow light to travel therethrough. Additionally or alternatively, the transparent component can be made of materials that allow electron beam, infrared, ultraviolet, electromagnetic wave, fluorescence, or the like to travel therethrough. The transparent component can have various shapes, such as a rectangular shape, a round shape, a polygon shape, an oval shape, an irregular shape, or the like. The size of the transparent component can be configured to expose the entire biopsy sample to the imaging device.

In this example, the biopsy needle can be a sheathed needle which includes a sheath and a needle. The sheath can be configured to accommodate the needle. The needle can be configured to collect and preserve biopsy sample(s) from a patient or a recipient. In some examples, a recipient can be an animal, a human, a plant, a living object, or the like.

At operation 1004, the process can include staining cells in the sample by introducing one or more tissue stains into the container. The tissue stains can enhance contrast for morphological imaging and molecular imaging. In some examples, the tissue stains can stay in the container for a period suitable to stain the biopsy sample(s). In some examples, the tissue stains can include different types of stains that are useful for diagnosing different types of pathologies, such as different types of cancers.

In some examples, the tissue stains can be introduced into the container via one or more ports. In some examples, the tissue stains can be introduced into the container at a given fluidic rate. In some examples, a respective port can have a valve configured to open, close, and/or control the fluidic rate of the tissue stains. In some examples, the tissue stain can be dripped or sprayed directly onto the tissue surfaces with a container that may not be water-tight.

At operation 1006, the process can include rinsing the tissue stains from the tissue which may include removal from the container. In some examples, after the biopsy sample(s) is stained, the extra tissue stains can be discharged from the container via one or more ports. In some examples, the one or more ports can include exit ports and entrance ports, where the exit ports are configured to discharge the tissue stains out of the container, and the entrance ports are configured to introduce the staining solution into the container. Additionally or alternatively, the one or more ports can be used for both introducing and discharging the staining solution into/from the container. Alternatively, a port may be used to spray a rinsing solution (such as phosphate buffered saline) onto the tissue surfaces and an exit port may be used to drain the solution like from a shower.

At operation 1008, the process can include compressing, by the window, the sample in the container. The imaging device can be controlled to press, via a support assembly, against the window so as to compress the biopsy sample(s). In some examples, in response to the pressing force applied by the imaging device via the support assembly, the flexible component can deform, and the transparent component can be displaced to contact the biopsy sample(s) to compress the biopsy sample(s). Additional details regarding the window with a flexible component are described with respect to FIG. 2A, FIG. 2B, FIG. 3A to FIG. 3E, FIG. 4A to FIG. 4E, FIG. 5, and FIG. 6. Alternatively, the window can be made of rigid and optically clear material without the flexible component. Additional details regarding the window without a flexible component are described with respect to FIG. 7A, FIG. 7B, FIG. 8, FIG. 9A, and FIG. 9B.

At operation 1010, the process can include capturing, via an imaging device, images of the sample through the window. In some examples, the imaging device can be controlled to capture images of the biopsy sample(s) in the container via the transparent component. In some examples, the imaging device can have a focus plane on the surface of the transparent component or the surface of the biopsy sample(s). The focus plane can be adjusted as needed. In some examples, the imaging device can include a wide field-of-view (FOV) objective lens. In some examples, the wide FOV objective lens can be used for a low-magnification wide FOV imaging. Additionally or alternatively, a high-resolution objective lens can be used, while the FOV thereof can be much smaller than the wide FOV objective lens. In some examples, the imaging device can move along various directions (e.g., horizontally, vertically, laterally, or the like) while applying the pressing force to the surface of the transparent component. The imaging device can capture images of different parts of the biopsy sample(s) while moving along various directions to scan the biopsy sample(s).

In some examples, after taking the images of the biopsy sample(s), the imaging device can be lifted from the transparent component of the window. As the force applied to the transparent component goes away, the transparent component can go back to the original position, and the flexible component can recover to the original shape and position. Additional details regarding the window with a flexible component are described with respect to FIG. 2A, FIG. 2B, FIG. 3A to FIG. 3E, FIG. 4A to FIG. 4E, FIG. 5, and FIG. 6. Additionally or alternatively, for the window without a flexible component, additional details are described with respect to FIG. 7A and FIG. 7B, FIG. 8, FIG. 9A and FIG. 9B.

In some examples, the system for imaging biopsy samples can also include a light source configured to emit light through the transparent component. Examples of the light source can include, but are not limited to, flashlight, lantern, limelight, laser device, light-emitting diode (LED), epi-illumination device, excitation light device, or the like.

At operation 1012, the process can include flushing the container to discharge the sample. In some examples, wash fluid can be introduced into the container to flush the container to discharge the biopsy samples. In some examples, the wash fluid can be introduced into the container via an inlet. The wash fluid can be introduced into the container at a given fluidic rate. Then, the wash fluid can wash away the biopsy samples through an outlet. Extra wash fluid can be discharged from the container after the biopsy samples are flushed off.

At operation 1014, the process can include storing the images in a storage component, and transmitting the images to a remote computing system. The storage component includes volatile and non-volatile, removable, and non-removable media implemented in any method or technology for the storage of information, such as computer-readable instructions, data structures, program modules, or other data. The storage component includes, but is not limited to, Phase Change Memory (PRAM), Static Random-Access Memory (SRAM), Dynamic Random-Access Memory (DRAM), other types of Random-Access Memory (RAM), Read Only Memory (ROM), Electrically Erasable Programmable ROM (EEPROM), flash memory or other memory technology, Compact Disk ROM (CD-ROM), Digital Versatile Discs (DVD) or other optical storage, magnetic cassettes, magnetic tape, magnetic disk storage or other magnetic storage devices, or any other medium that can be used to store information for access by a computing device.

The system for imaging biopsy samples can be in communication with a remote computing system, for example, via one or more networks. For example, the system for imaging biopsy samples can transmit data to the remote computing system. The data can include the images of the biopsy samples(s) 110 captured by the imaging device, the assistant diagnostic information generated by the image analysis component, and other data. In some examples, other data can include, but is not limited to, time data, position data of the imaging device, position data of the transportation state, stain type, or the like.

The networks can be any type of wireless network or other communication network known in the art. Examples of the networks 130 include the Internet, an intranet, a wide area network (WAN), a local area network (LAN), and a virtual private network (VPN), cellular network connections, and connections made using protocols such as Institute of Electrical and Electronics Engineers (IEEE) standards, including 802.11a, b, g, n, and/or ac. The 802.11a, b, g, n, and/or ac are IEEE standards used for wireless routers, Wi-Fi access points, and Wi-Fi in portable devices.

At operation 1016, the process can include providing, using a machine learned model, assistant diagnostic information regarding whether the cells include pathologies, such as at least one cancer cell. In some examples, system for imaging biopsy samples can implement a machine learned model to analyze the images of the biopsy sample(s). In some instances, the machine learned model can include any models, algorithms, and/or machine learning algorithms. For example, the machine learned model can be implemented as a neural network. As described herein, an exemplary neural network is a biologically inspired algorithm that passes input data through a series of connected layers to produce an output. Each layer in a neural network can also include another neural network, or can include any number of layers (whether convolutional or not). As can be understood in the context of this disclosure, a neural network can utilize machine learning, which can refer to a broad class of such algorithms in which an output is generated based on learned parameters. Although discussed in the context of neural networks, any type of machine learning can be used consistently with this disclosure.

The remote computing system is configured to receive, display, and/or analyze the data. For example, the remote computing system can include a display configured to visually output images of the biopsy sample(s) based on the data. The remote computing system can also display the assistant diagnostic information based on the data. The remote computing system can be associated with a user. In some examples, the display can display a Ul which may facilitate an interaction between the remote computing system and the user. The user can view the images of the biopsy sample(s), the assistant diagnostic information, and other data. The user can also provide feedback, diagnostic comments, opinions, or the like via the Ul through the remote computing system. In some examples, the user can be a pathologist, an oncologist, a cytologist, a physician, or the like. For example, the user (e.g., a pathologist, an oncologist, a cytologist, a physician, or the like) can provide a comment that the sample includes pathologies, such as at least one cancer cell.

The techniques described herein may be performed by various devices in a medical environment, such as bedside devices, care on point devices, test systems, and so forth.

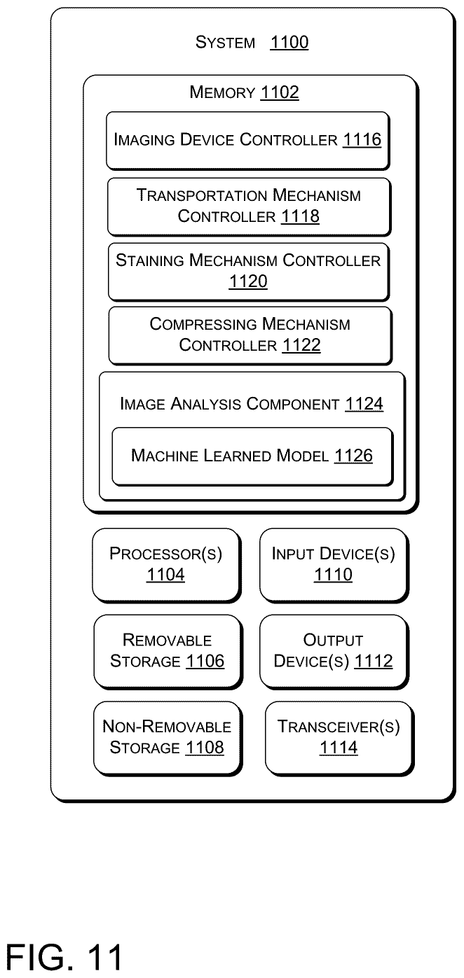

FIG. 11 illustrates an example system 1100 configured to enable and/or perform the some or all of the functionality discussed herein. Further, the system 1100 can be implemented as one or more server computers, a network element on dedicated hardware, as a software instance running on dedicated hardware, or as a virtualized function instantiated on an appropriate platform, such as cloud infrastructure, and the like. It is to be understood in the context of this disclosure that the system 1100 can be implemented as a single device or as a plurality of devices with components and data distributed among them.

As illustrated, the system 1100 includes a memory 1102, one or more processors 1104, a removable storage 1106, a non-removable storage 1108, one or more input devices 1110, one or more output devices 1112, and one or more transceiver(s). In various embodiments, the memory 1102 can be volatile (including a component such as Random Access Memory (RAM)), non-volatile (including a component such as Read Only Memory (ROM), flash memory, etc.), or some combination of the two. The memory 1102 may include various components that are executable by the processor(s) 1104 to perform functions, such as an imaging device controller 1116, a transportation mechanism controller 1118, a staining mechanism controller 1120, a compressing mechanism controller 1122, and an image analysis component 1124. Note that these components are examples rather than limitations, and the system 1100 can include other components as necessary.

The imaging device controller 1116 is configured to control an imaging device to capture images. The imaging device controller can also be configured to move the imaging device in various directions to adjust the position of the imaging device. The imaging device controller can also be configured to control the imaging device to apply a pressing force to a window which is configured to compress one or more biopsy samples. The imaging device controller can also be configured to control the force applied to the window with sensors (such as vision sensors, displacement sensors, and/or force sensors).

The transportation mechanism controller 1118 is configured to control a transportation mechanism to move along various directions (e.g., horizontally, vertically, diagonally, or the like). In some examples, the container and/or the biopsy device can be placed on the transportation mechanism to move together with the transportation mechanism.

The transportation mechanism controller can be configured to control the transportation mechanism to change the position of the container and/or the biopsy device for staining and/or imaging the biopsy sample(s).
The staining mechanism controller 1120 is configured to control the staining mechanism to stain the biopsy sample(s). For example, the staining mechanism controller can be configured to control the staining mechanism to provide the staining solution to the container via one or more ports (not shown) at a given fluidic rate. The staining mechanism controller can also be configured to control the staining mechanism to discharge the staining solution from the container 106 after the biopsy sample(s) is stained. Additionally or alternatively, the staining mechanism controller dispenses the staining solution (such as in droplets) to the biopsy sample(s) rather than immersing the biopsy sample(s). Additional details are described with respect to FIG. 9A.

The image analysis component 1124 is configured to provide assistant diagnostic information based, at least in part, on the images of the biopsy sample(s). In some examples, the assistant diagnostic information can include information regarding whether the cells of the biopsy sample(s) include different types of pathologies, such as at least one cancer cell. In some examples, the image analysis component 1124 can implement a machine learned model 1126 to analyze the images of the biopsy sample(s). In some instances, the machine learned model 1126 can include any models, algorithms, and/or machine learning algorithms. For example, the machine learned model 1126 can be implemented as a neural network. As described herein, an exemplary neural network is a biologically inspired algorithm that passes input data through a series of connected layers to produce an output. Each layer in a neural network can also include another neural network, or can include any number of layers (whether convolutional or not). As can be understood in the context of this disclosure, a neural network can utilize machine learning, which can refer to a broad class of such algorithms in which an output is generated based on learned parameters.

In some embodiments, the processor(s) 1104 includes a Central Processing Unit (CPU), a Graphics Processing Unit (GPU), or both CPU and GPU, or other processing units or components known in the art.
The system 1100 can also include additional data storage devices (removable and/or non-removable) such as, for example, magnetic disks, optical disks, or tape. Such additional storage is illustrated in FIG. 11 by removable storage 1106 and non-removable storage 1108. Tangible computer-readable media can include volatile and nonvolatile, removable, and non-removable media implemented in any method or technology for the storage of information, such as computer-readable instructions, data structures, program modules, or other data. The memory 1102, removable storage 1106, and non-removable storage 1108 are all examples of computer-readable storage media. Computer-readable storage media include, but are not limited to, RAM, ROM, EEPROM, flash memory or other memory technology, CD-ROM, Digital Versatile Discs (DVDs), Content-Addressable Memory (CAM), or other optical storage, magnetic cassettes, magnetic tape, magnetic disk storage or other magnetic storage devices, or any other medium which can be used to store the desired information and which can be accessed by the system 1100. Any such tangible computer-readable media can be part of the system 1100.
The system 1100 also can include input device(s) 1110, such as a keypad, a cursor control, a touch-sensitive display, a voice input device, etc., and output device(s) 1110 such as a display, speakers, printers, etc. These devices are well-known in the art and need not be discussed at length here. In particular implementations, a user can provide input to the system 1100 via a user interface associated with the input device(s) 1110 and/or the output device(s) 1112.
As illustrated in FIG. 11, the system 1100 can also include one or more wired or wireless transceiver(s) 1114. For example, the transceiver(s) 1114 can include a Network Interface Card (NIC), a network adapter, a LAN adapter, or a physical, virtual, or logical address to connect to the various base stations or networks contemplated herein, for example, or the various user devices and servers. To increase throughput when exchanging wireless data, the transceiver(s) 1114 can utilize Multiple-Input/Multiple-Output (MIMO) technology. The transceiver(s) 1114 can include any sort of wireless transceiver capable of engaging in wireless, Radio Frequency (RF) communication. The transceiver(s) 1114 can also include other wireless modems, such as a modem for engaging in Wi-Fi, WiMAX, Bluetooth, or infrared communication.
In some implementations, the transceiver(s) 1114 can be used to communicate between various functions, components, modules, or the like, that are included in the system 1100. For instance, the transceiver 1114 may facilitate communications between the system 1100 and other devices such as a remote computing system 128 described with respect to FIG. 1.

The example systems and methods of the present disclosure overcome various deficiencies of known prior art devices. Other embodiments of the present disclosure will be apparent to those skilled in the art from consideration of the specification and practice of the disclosure contained herein. It is intended that the specification and examples be considered as an example only, with a true scope and spirit of the present disclosure being indicated by the following claims.

### EXAMPLE CLAUSES

Any of the example clauses in this section may be used with any other of the example clauses and/or any of the other examples or embodiments described herein.

A: A system including: a window including an inflexible and optically clear material, the window being configured to compress a sample disposed in a biopsy needle; and an imaging device configured to press the window to compress the sample, the imaging device being configured to capture one or more images of the sample through the window.

B: The system of clause A, wherein the sample includes a soft tissue sample.

C: The system of either clause A or B, wherein the imaging device includes a support assembly configured to press the window via a support component, a focal length of the imaging device being at the window when the support assembly is in contact with the window via the support component.

D: The system of any one of clauses A-C, further including: a dispenser configured to dispense a tissue stain, the tissue stain being configured to stain cells in the sample.

E: The system of clause D, wherein the tissue stain includes at least one of hematoxylin, eosin, Hoechst, or rhodamine B.

F: The system of any one of clauses A-E, further including: a microfluidic circuit including: a first reservoir storing a tissue stain configured to stain cells; a second reservoir storing a wash fluid; at least one waste receptacle; a container configured to receive the biopsy needle and including the window; and at least one pump configured to: move the tissue stain from the first reservoir into the container; move the tissue stain out of the container and into the at least one waste receptacle; move a wash fluid from the second reservoir into the container; and move the wash fluid out of the container and into the at least one waste receptacle.

G: The system of clause F, wherein the wash fluid includes at least one of saline solution, formalin, or an alcohol chemical fixation.

H: The system of any one of clauses A-G, further including: a processor configured to determine, based on the one or more images, whether the sample includes at least one cancer cell.

I: A system including: a container configured to receive a biopsy needle, the container including a window configured to optically expose a sample disposed in the biopsy needle; and an imaging device arranged proximate to the window, the imaging device being configured to capture images of the sample through the window.

J: The system of clause I, wherein the container further includes: a flexible frame coupled to a border of the window, the window being transparent.

K: The system of clause J, wherein the flexible frame includes an elastic material.

L: The system of any one of clauses I-K, wherein the window is configured to compress the sample in the container.

M: The system of any one of clauses I-L, further including a light source configured to emit light through the window.

N: The system of any one of clauses I-M, further including: a stage configured to accommodate the container and to move the container with respect to the imaging device.

O: The system of any one of clauses I-N, further including: a dispenser configured to dispense a tissue stain; a waste receptacle; and at least one pump configured to: move the tissue stain from the dispenser into the container; and move the tissue stain from the container into the waste receptacle.

P: The system of any one of clauses I-O, wherein the imaging device includes at least one of an optical microscope, an electron microscope, a stereomicroscope, or a fluorescence microscope.

Q: The system of any one of clauses I-P, further including: a sealing component disposed between the biopsy needle and the container, the sealing component being configured to seal fluid inside the container.

R: The system of any one of clauses I-Q, wherein the sample includes a soft tissue sample.

S: A method, including: receiving, in a container including a window, a sample from a biopsy needle, the window including an inflexible and optically clear material; staining cells in the sample by introducing one or more tissue stains into the container; compressing, via the window, the sample in the container; and capturing, via an imaging device, images of the sample through the window.

T: The method of clause S, wherein compressing, via the window, the sample in the container includes: stretching a flexible frame coupled to an edge of the window.

U: The method of either clause S or T, wherein compressing, via the window, the sample in the container includes: pressing, by the imaging device, the window such that the inflexible and optically clear material of the window contacts the sample to compress the sample.

V: The method of any one of clauses S-U, wherein compressing, by the window, the sample in the container includes: pressing, by a compressing component, a slide component of the container, the slide component being coupled to a flexible frame of the container.

W: The method of any one of clauses S-V, wherein capturing, via the imaging device, images of the sample through the window of the container includes: moving the imaging device along a direction parallel to the window to capture the images of the samples.

X: The method of any one of clauses S-W, further including: flushing the container to discharge the sample.

Y: The method of any one of clauses S-X, further including: emitting, by a light source, light through the window.

Z: The method of any one of clauses S-Y, wherein capturing, via the imaging device, images of the sample through the window of the container includes: moving, by a transportation mechanism, the container with respect to the imaging device.

AA: The method of any one of clauses S-Z, further including: storing the images in a storage component; and transmitting the images to a remote computing system.

AB: The method of any one of clauses S-AA, wherein staining cells in the sample by introducing one or more tissue stains into the container includes: introducing, via at least one port, a first tissue stain into the container to stain the cells; discharging, via the at least one port, the first tissue stain from the container; introducing, via the at least one port, a second tissue stain into the container to stain the cells; and discharging, via the at least one port, the second tissue stain from the container.

AC: The method of any one of clauses S-AB, further including: introducing a fixation reagent into the container to fix the sample.

AD: The method of any one of clauses S-AC, further including: providing, using a machine learned model, assistant diagnostic information based, at least in part, on the images of the sample, the assistant diagnostic information including information regarding whether the cells include at least one cancer cell.

AE: One or more computer-readable media storing instructions, which when executed by one or more processors, cause the one or more processors to perform operations including: causing at least one pump to introduce a tissue stain to a sample disposed in a biopsy needle, thereby staining cells in the sample; causing an actuator to compress the sample by moving a window, the window including an inflexible and optically clear material; and causing an imaging device to capture images of the sample through the window.

While the example clauses described above are described with respect to particular implementations, it should be understood that, in the context of this document, the content of the example clauses can be implemented via a method, device, system, a computer-readable medium, and/or another implementation. Additionally, any of the examples A-AE may be implemented alone or in combination with any other one or more of the examples A-AE.

### CONCLUSION

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the implementation to the specified elements, steps, ingredients, or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; +11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

“Specifically binds” refers to an association of a binding domain (of, for example, a CAR binding domain or a nanoparticle selected cell targeting ligand) to its cognate binding molecule with an affinity or Ka (i.e., an equilibrium association constant of a particular binding interaction with units of 1/M) equal to or greater than 105 M-1, while not significantly associating with any other molecules or components in a relevant environment sample. “Specifically binds” is also referred to as “binds” herein. Binding domains may be classified as “high affinity” or “low affinity”. In particular embodiments, “high affinity” binding domains refer to those binding domains with a Ka of at least 107 M-1, at least 108M-1, at least 109 M 1, at least 1010 M-1, at least 1011 M-1, at least 1012 M-1, or at least 1013 M-1. In particular embodiments, “low affinity” binding domains refer to those binding domains with a Ka of up to 107 M-1, up to 106 M-1,and up to 105 M-1. Alternatively, affinity may be defined as an equilibrium dissociation constant (Kd) of a particular binding interaction with units of M (e.g., 10-5 M to 10-13 M). In certain embodiments, a binding domain may have “enhanced affinity,” which refers to a selected or engineered binding domains with stronger binding to a cognate binding molecule than a wild type (or parent) binding domain. For example, enhanced affinity may be due to a Ka (equilibrium association constant) for the cognate binding molecule that is higher than the reference binding domain or due to a Kd (dissociation constant) for the cognate binding molecule that is less than that of the reference binding domain, or due to an off-rate (Koff) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. No. 5,283,173, U.S. Pat. No. 5,468,614, or the equivalent).

Unless otherwise indicated, the practice of the present disclosure can employ conventional techniques of immunology, molecular biology, microbiology, cell biology, and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

1. A system comprising:
a window comprising an inflexible and optically clear material, the window being configured to compress a sample disposed in a biopsy needle; and
an imaging device configured to press the window to compress the sample, the imaging device being configured to capture one or more images of the sample through the window.

2. The system of claim 1, wherein the sample comprises a soft tissue sample.

3. The system of claim 1, wherein the imaging device comprises a support assembly configured to press the window via a support component, a focal length of the imaging device being at the window when the support assembly is in contact with the window via the support component.

4. The system of claim 1, further comprising:
a dispenser configured to dispense a tissue stain, the tissue stain being configured to stain cells in the sample; and/or
a processor configured to determine, based on the one or more images, whether the sample comprises at least one cancer cell.

5. (canceled)

6. The system of claim 1, further comprising:
a microfluidic circuit comprising:
a first reservoir storing a tissue stain configured to stain cells;
a second reservoir storing a wash fluid;
at least one waste receptacle;
a container configured to receive the biopsy needle and comprising the window; and
at least one pump configured to:
move the tissue stain from the first reservoir into the container;
move the tissue stain out of the container and into the at least one waste receptacle;
move the wash fluid from the second reservoir into the container; and
move the wash fluid out of the container and into the at least one waste receptacle.

7. The system of claim 6, wherein the wash fluid comprising at least one of saline solution, formalin, or an alcohol chemical fixation.

8. (canceled)

9. A system comprising:
a container configured to receive a biopsy needle, the container comprising a window configured to optically expose a sample disposed in the biopsy needle; and
an imaging device arranged proximate to the window, the imaging device being configured to capture images of the sample through the window.

10. The system of claim 9, wherein the container further comprises:
a flexible frame coupled to a border of the window, the window being transparent.

11. The system of claim 10, wherein the flexible frame comprises an elastic material.

12. The system of claim 9, wherein the window is configured to compress the sample in the container, and/or
wherein the imaging device comprises at least one of an optical microscope, an electron microscope, a stereomicroscope, or a fluorescence microscope.

13. The system of claim 9, further comprising:
a light source configured to emit light through the window; and/or
a stage configured to accommodate the container and to move the container with respect to the imaging device.

14. (canceled)

15. The system of claim 9, further comprising:
a dispenser configured to dispense a tissue stain;
a waste receptacle; and
at least one pump configured to:
move the tissue stain from the dispenser into the container; and
move the tissue stain from the container into the waste receptacle.

16. (canceled)

17. The system of claim 9, further comprising:
a sealing component disposed between the biopsy needle and the container, the sealing component being configured to seal fluid inside the container.

18. (canceled)

19. A method, comprising:
receiving, in a container comprising a window, a sample from a biopsy needle, the window comprising an inflexible and optically clear material;
staining cells in the sample by introducing one or more tissue stains into the container;
compressing, via the window, the sample in the container; and
capturing, via an imaging device, images of the sample through the window.

20. The method of claim 19, wherein compressing, via the window, the sample in the container comprises at least one of:
stretching a flexible frame coupled to an edge of the window;
pressing, by the imaging device, the window such that the inflexible and optically clear material of the window contacts the sample to compress the sample; or
pressing, by a compressing component, a slide component of the container, the slide component being coupled to a flexible frame of the container.

21. (canceled)

22. (canceled)

23. The method of claim 19, wherein capturing, via the imaging device, images of the sample through the window of the container comprises:
moving the imaging device along a direction parallel to the window to capture the images of the sample; and/or
moving, by a transportation mechanism, the container with respect to the imaging device.

24. The method of claim 19, further comprising at least one of:
flushing the container to discharge the sample;
emitting, by a light source, light through the window; or
introducing a fixation reagent into the container to fix the sample.

25. (canceled)

26. (canceled)

27. The method of claim 19, further comprising:
storing the images in a storage component; and
transmitting the images to a remote computing system.

28. The method of claim 19, wherein staining cells in the sample by introducing one or more tissue stains into the container comprises:
introducing, via at least one port, a first tissue stain into the container to stain the cells;
discharging, via the at least one port, the first tissue stain from the container;
introducing, via the at least one port, a second tissue stain into the container to stain the cells; and
discharging, via the at least one port, the second tissue stain from the container.

29. (canceled)

30. The method of claim 19, further comprising:
providing, using a machine learned model, assistant diagnostic information based, at least in part, on the images of the sample, the assistant diagnostic information comprising information regarding whether the cells comprise at least one cancer cell.
